(19) United States
(12) Patent Application Publication
(10) Pub. No.: US 2021/0007360 A1
(43) Pub. Date: Jan. 14, 2021

(54) SOLUTIONS COMPRISING OZONIZED OIL

(71) Applicant: Ahmet Ümit SABANCI, Bursa (TR)

(72) Inventor: Ahmet Ümit SABANCI, Bursa (TR)

(21) Appl. No.: 17/040,825

(22) PCT Filed: Mar. 29, 2019
(86) PCT No.: PCT/TR2019/050209

§ 371 Date: Sep. 23, 2020

(30) Foreign Application Priority Data
Mar. 29, 2018 (TR) 2018/04452
Mar. 29, 2019 (TR) 2019/04790

(51) Int. Cl. A01N 59/00; A01N 25/04; A61K 33/00; A61K 8/22; C11D 3/48; C05G 5/23; A61K 9/00; A61Q 7/00; A61K 8/06; A61K 9/08; A23K 20/158; A23L 3/3472; A23D 7/005; A23K 20/20; A23L 3/358

(52) U.S. Cl. CPC A01N 59/00; A23L 3/358; A61K 33/00; A61K 8/22; C11D 3/48; C05G 5/23; A61K 9/0043; A61K 9/0019; A61Q 7/00; A61K 8/06; A61K 9/0048; A61K 9/0031; A61K 9/08; A23K 20/158; A23L 3/3472; A23D 7/0053; A23K 20/20; A01N 25/04

(57) ABSTRACT

Disclosed are solutions having ozone in designated doses, and ozonized oil in nano or micron scales, and also methods of production and to the use of such solutions.

### TECHNICAL FIELD

The invention relates to solutions comprising ozonized oil, to their production methods and to the applications/uses of these solutions.

The invention relates in particular to solutions comprising ozonized oil in nano or micron scales, which comprising ozone in designated doses, to their methods of production and to the use of such solutions.

### PRIOR ART

The allotrope of oxygen, ozone (O3) is a compound of three oxygen atoms. Ozone is a colorless, sharp-odorous gas with high oxidation power. When the usage of ozone is examined, a wide variety of applications are encountered. Ozone is used as a disinfectant and deodorant in water and air. Since the oxidation power is high, it removes the organic molecules and microorganisms that pollute the environment. Accordingly, it is used as a disinfectant in the food industry and for the long term storage of foods. It is used in a variety of fields such as for infections, circulatory disorders, joint diseases, intestinal diseases, skin diseases, etc. in medicine, for the purpose of protecting animal health and for the treatment of diseases in veterinary medicine, for skin and hair products in cosmetics, and for bleaching in textile. Ozone occurs in nature through ultraviolet rays of sun or lightnings. In the current technique, ozone can be produced via various production technologies. Ozone generators are generally used for this purpose.

The use of ozone in the field of healthcare is called ozone therapy. The main ozone therapy methods in the current technique are as follows: major autohemotherapy, minor autohemotherapy, ozone enriched water, ozone/oxygen gas mixture injection directly into the joints and tissues, rectal and vaginal applications, external therapy and ozone enriched oil applications.

Since ozone is an active molecule, it loses its effect in a short time and turns into oxygen. Ozone is a non-stable gas that must be used at a time because it cannot be stored and has a 40-minute half life at 20° C. Therefore, it is generally used where it is produced and in a short period of time. In addition, the usage areas of ozone therapy are limited and the costs are quite high due to the fact that it should be performed by experts and the environments such as hospitals or clinics are required for the application. Also, some of the disadvantages of the ozone therapy methods are that ozone therapy methods are not practical and cannot be applied by individuals in anytime, anywhere.

In order to promote the use of ozone for therapeutic purposes and to make it salable in pharmacies, the oils that are known as the best movers of ozone and as keeping it stable for a long time are used. For this purpose, ozonized oils are obtained by passing the ozone gas through the oils. Ozone gas is widely used in the petrochemical and pharmaceutical industries due to its high reactivity between organic bonds. Ozone gas is called ozonide when it becomes unstable following the oleic acid reaction. In this reaction, defined as the Criegee reaction, ozonide is converted into trioxolane-like carbonyl fragments, aldehyde, and peroxides. The use of herbal oils by ozonization is known in the prior art.

In the patent document US2006074129, a method for obtaining ozonized oils and vegetable fats and use of said products for pharmaceutical and cosmetic purposes is explained. The emulsified vegetable oil or fat is driven to react with ozone in a bubbling reactor, covered by a jacket that makes possible to control the reaction's temperature. Ozone reacts with the double bonds present in vegetable oil or fat, giving rise to the formation of different peroxidic species, responsible for the biological activity of those products. The ozonized vegetable oils and fats in those conditions are applied with satisfactory and advanced results to the treatment of diseases in diverse medical specialties, such as ophthalmology, dentistry, dermatology, gastroenterology, gynecology, parasitology, and others.

In the patent document EP0235528, a process for producing stable ozonized oils from unsaturated vegetable oils by introducing an ozone-oxygen mixture into the oil to saturation is explained. In this method, ozone gas is very slowly passed through the oil at a certain speed and time. Unsaturated fatty acids found in vegetable oils react with ozone and organic peroxides occur. Following ozonization, an extraction treatment in an acid environment in the presence of a redox system to a radical reaction in order to separate short chain aldehydes from the ozonized oil is applied. Saturation of vegetable oils with ozone causes them to solidify. Therefore, the ozonized oils obtained with this method can be applied only through the skin.

The document numbered as TR2010/02961 describes the water-based personal care and cleaning liquid comprising the ozone derivative of vegetable oils and the method for obtaining it. The said personal care and cleaning fluid is derived from the addition and mixing of the vegetable oil ozone derivative into a mixture comprising water and at least one emulsifier. In the mixture of the invention, ozonide, water and emulsifier are mixed between 35 to 55° C. and petroleum derivatives are not used. In addition, to obtain the personal care and cleaning fluids of the invention, only ozonized herbal oils are used. In addition, the mixtures obtained with this method are used only for cosmetic purposes.

The document no. EP3311800A1 discloses a composition comprising microstructures for the controlled release of active agents, in particular, ozonized oil. Said microstructures are coated with a cationic surfactant comprising ammonium group polysaccharide. However, there is no information about the amount of ozone gas and the size distribution of microstructures in the samples where ozonized oils are used. There is only an expression that 5% ozonide in solution was used. The amount of ozone gas contained in the composition is a factor that determines the effectiveness. In the said application, ozone can only be used in cosmetics and textile industry.

Application no. U.S. Pat. No. 4,983,637A refers to a method for treating a viral infection of HIV. In this method, the use of ozonides of terpenes as antiviral agents is described. It is stated that the amount of ozone formed as a result of ozonization of terpene is 2-6% and it is said that it should be mixed with polyethylene glycol at certain rates and should not be combined with water. It is written that diluting the mixture with water would reduce the effectiveness and indicated that it should be mixed with the physiological saline solution at the application, but no amount of physiological saline solution was given. This does not explain the dose. However, in order to be injected as a drug, the amount of all the ingredients must be stated. It has been reported that between 1 mg and 10 g per day should be used twice a day through systemic use and should be used every day. However, ozonide is an oil that must be used with caution because of its burning properties. This form can only be used uncontrollably on the skin. At the same time, the absence of nano and micron sizes will cause absorption disorders. Terpen ozonide is intended for use only for the virus treatments.

In the review article titled entitled as ‘Particle size in parenteral fat emulsions, what are the true limitations?’ published by V. S. Koster et al. in 1996, particle sizes in parental oil emulsions were discussed. Although the safe maximum diameter to prevent lung embolism in parental emulsions is considered to be less than 5 micrometers, pathologies with larger diameters have been detected in emulsions that have been used for several years without significant adverse reactions. Therefore, it has been concluded that the requirements for the limitation of particle size in oil emulsions should be revised.

Ozonized oils facilitate topical application of ozone therapy. However, some difficulties are experienced in the personal and domestic use of the ozonized oils available. Since ozone is a reactive molecule, it is very important to pay attention to the use of ozone for medical purposes. The oils used should preferably be compatible with the tissue and contain the appropriate dose of ozone. In the present technique, ozonized oils are generally applied epidermally (administrated cutaneously). Applications by injection are usually performed by mixing the appropriate mixtures of ozone/oxygen gases with the blood of the patient to be treated and then returning the blood mixture to the patient. Due to the invention, the mixtures/solutions obtained by the ozonization of the injectable oils can be used by injection. In addition, there are problems in adjusting the density of the oils used in the current technique and in the absorption of the said oils. While the use of dense oils damages tissues, the fat being in large particles leads to problems of absorption. However, the oils that are ozonized by the production method of the invention are lysed into nano scales or micron sizes in water. Thus, the risk of tissue damage and absorption problems are overcome.

The injectable oils have been used for years to inject fat-soluble vitamins A, D, E, K and steroids into the tissue. Due to the low side effects of injectable oils, with the ozonization of the same oils and the formation of ozonized oil or ozonides having water-soluble nano or micron particle size, these oils, which are easily injected and can be measured in milligrams, are intended to be widely used as they are sold in pharmacies without the need for an ozonizer. At the same time, with this application, new usage areas of ozone are created.

In the current technique, paraffin and similar oils are squeezed onto the fruit and vegetables to make them look brighter. This process is harmful to health. The use of ozonized oil solutions created as a result of the ozonization of vegetable oils in diluted doses for the same purpose prolongs the shelf life of fruits and vegetables. Due to the oil content, these solutions make them look bright and fresh. Due to water content, drying of them is prevented. Thanks to the present invention, the solution comprising ozonized oil delayed the process of decay by preventing bacteria and fungus production with the thin layer formed by the fruits and vegetables on which it is squeezed. Similarly, the packaging of meat by passing it through the ozone gas increases shelf life by two times. Squeezing of ozonized oil-comprising solutions formed by ozonization of vegetable oils in a thin layer in dilute doses during the packaging of white and red meat ensures that the meats seem fresh and alive, and prevents them from drying due to its water content. It also prevents bacterial, viral and fungal contamination and prolongs the shelf life. There is no harm in the consumption of these foods. As it is known, when ozone and solutions comprising these oils are ingested, they have healing effects on the digestive system. The use of solutions comprising ozonized oil as stabilizers in order to prevent decay of the packaged foods and beverages makes it both harmless and a useful additive for the digestive system and general body health. Plant spraying is costly and has a negative effect on both people and the environment due to the spread of chemical wastes to nature. The application made with solutions comprising ozonized oil is completely natural, it does not harm the plant and prevents harmful bacteria and fungi from forming cavities. In order to protect ornamental plants and improve the health of the leaf, the application of ozone-comprising oil solutions to the leaves and the trunk/body provides an enhancing effect on plant health.

As a result, due to the above-mentioned drawbacks and the inadequacy of the existing solutions, an improvement in the technical field has been required.

### THE PURPOSE OF INVENTION

The invention is inspired by the existing circumstances and aims to solve the above-mentioned drawbacks.

The main purpose of the invention is to enable ozonized oils comprising ozone in designated doses to be used in many areas by making available their chemical degradation into nano or micron sizes in water. It is especially to overcome the problems of ozone concerning the use of it in designated doses to avoid damaging tissues and its absorption.

Another purpose of the invention is to prepare solutions comprising ozone or ozonized oil in nano or micron sizes obtained by ozonizing the oils used for injection purposes and to ensure that they can be injected into the tissues individually or within a mixture/solution prepared with the appropriate substances.

Another purpose of the invention is to reduce the side effects of each oil in mixtures comprising more than one oil (multiple mixtures) in the solutions comprising ozonized oil or ozonide. In all applications, the solutions comprising the ozonized oil or ozonide according to the invention can be used on an individual basis or as combinations by mixing.

Another purpose of the invention is to ensure the safe use of the methods of the invention due to the measurability of the amount of ozone obtained by measuring ozonized oil in grams. Because, as a result of ozonization, oils are increased by weights.

Another purpose of the invention is to dissolve ozonized oils or ozonides formed with injectable oils in water to increase the absorption of them in tissues, thereby making it possible to use ozonized oils or ozonides injectable in the form of degraded to 100-400 microns or to the particle size of 1 nanometer by using ultrasonic methods or using high-speed motors without requiring heat treatment. This new product is called solution with ozonized oil (or ozonide solution). Using various methods, ozonized oil or ozonides can be degraded into particle sizes in a wide range from 1 nanometer to 400 micrometers.

Another purpose of the invention is to standardize the ozone machine for the amount of ozone that the oils obtain and to provide the use of devices that calculate the current and ozone-oxygen content, in order to generate ozone-oxygen in the amount of ozone. As ozone and oxygen amounts produced by ozone devices are in different doses, it is aimed to calculate the active substance in the ozonized solution precisely, based on the fact that the carrier is charged with more oxygen if the oxygen amount of the machine is more and with more ozone if the ozone amount of the machine is more.

Another purpose of the invention is to form ozonized oils or ozonides with high-resolution oil solutions in water, having a nano or micron particle size in the range of 1 nanometer to 400 micrometers. These ozonized oil or ozonide solutions are used for intramuscular, intraarticular, local effect, as a solution in wound treatment by local injection, as a nasal spray, ear drops, eye drops, in intra-oral wounds, for rectal washing and ozonization, rectal suppository, in vagina cleaning and intrauterine injections, and as a vaginal ovule and suppository, as a lubricant for urinary catheter into the bladder, as a local injection for the purpose of mesotherapy, in injections for the development of hair, and also in order to combine the effects of all kinds of external use, it may be used as a mixture with one or more injectable ozone oil, either alone or in combination with other drugs.

Another purpose of the invention is to ensure the use of ozonized oils or ozonides obtained as a result of the ozonization of all vegetable and chemical oils in various ratios depending on solution concentration or ratios in the chemical, cosmetics, agriculture, livestock sectors due to the chemical degradation of them into nano or micron particles.

Another purpose of the invention is that it can be used together with the drugs that need a prolonged effect time such as the local anesthetics, or anti-inflammatory drugs. The dense mixtures of ozonized oil solutions of the invention release ozone for times as long as 72 hours in intramuscular, intraarticular or local injections.

Another object of the invention is to provide the use of ozonized oils by allowing its combination with all fat-soluble vitamins and drugs.

The invention can be used to provide parenteral ozone when given slowly at low doses.

A further purpose of the invention is to provide high ozonization of fats such as cholesterol, triglyceride, phospholipid, to be administrated intraarterially in combination with vitamin K in arterial vein occlusion, and to open the vessel with the antioxidant effect of ozonized oil products adhering to the vessel wall.

Another purpose of the invention is to reduce the use of antibiotics in regional infections.

Since the injection of solutions comprising ozonized oil will be released slowly, it increases oxygenation and reduces the use of antibiotics. It can be applied as an injection into the abscess and periphery of it. In order to speed up the postoperative wound healing, it can also be injected to the surgical region.

Another purpose of the invention is to ensure that solutions comprising ozonized oil are used in wound treatment both by local injection and epidermally.

Another purpose of the invention is to increase the effects of soybean lecithin, phosphatidylcholine, alpha lipoic acid, and soy lecithin, which are used in regional mesotherapy for slimming purposes, by providing the ozonization of fats in nano or micron sizes.

Another purpose of the invention is to ensure the use of solutions comprising ozonized oil in mesotherapy alone or combined with other drugs for preventing hair loss.

Another purpose of the invention is to use the solutions comprising ozonized oil or ozonide that are formed by the ozonization of silicone oil and other ozonized oils or ozonides in skin mesotherapy.

Another purpose of the invention is to provide the application of solutions comprising ozonized oil in nano or micron sizes in the form of local fistule injections and regional injections in the treatment of mastitis.

Another purpose of the invention is to provide an enhanced antioxidant effect through regular injections of solutions comprising ozonized oil, and area of use in addition to cancer prevention and chemotherapy.

Another purpose of the invention is to make cancer tissue hypoxic by administering it to the cancerous tissue and surrounding area locally at high concentrations and high doses in cancer treatment.

Another purpose of the invention is to provide the cancer cell with a combination of chemotherapy drugs with ozonized oil solution for the treatment of cancer and with the consideration that the cancer cell as capable of taking up oxygen, to make it a selective drug that introduces the chemotherapeutic agent into the cell.

Another purpose of the invention is to provide the effect of major therapy with ozone applied 2-3 times a week for general purposes with the intramuscular or subcutaneous application.

Another purpose of the invention is to dissolve the ozone without reaching the lung tissue by using nano or micron size ozonized oil particles in water and by providing quick dissolving of the ozone in small doses in the intravenous way of the solutions comprising ozonized oil or ozonide. Thus, instead of blood transfusion, the use of solutions comprising ozonized oil is provided.

Another purpose of the invention is to provide the use of solutions comprising nano or micron-sized ozonized oils for the injections in the joint.

Another purpose of the invention is to provide more effective use of solutions comprising nano or micron-sized ozonized oils for the cervical disc and spinal disc hernia.

Another purpose of the invention is to provide the use of solutions comprising nano or micron-sized ozonized oils in fracture union and, in particular, in regional injections in fracture union.

Another purpose of the invention is to provide more effective use of solutions comprising nano or micron-sized ozonized oils in wound treatment and, in particular, in the regional injections or epidermal applications.

A further purpose of the invention is to provide the use of solutions comprising nano or micron-sized ozonized oil in all external forms and mucosal forms, together with silver nanoparticles or other metals that shows oligodynamic effects.

Another purpose of the invention is to provide more effective use of solutions comprising nano or micron-sized ozonized oils in the treatment of diseases such as injectable in the treatment of diseases such as tenosynovitis, fibromyalgia, myositis, plantar fasciitis.

Another purpose of the invention is to provide the use of solutions comprising nano or micron-sized ozonized oils as a lubricant in catheter applications.

A further purpose of the invention is to provide the intravesical use of solutions comprising nano or micron-sized ozonized oil in the treatment of cystitis, urethritis, ureteritis.

Another purpose of the invention is to provide the use of solutions comprising nano or micron-sized ozonized oils in the treatment of vulvovaginitis and vaginitis.

Another purpose of the invention is to provide the use of solutions particularly comprising nano or micron-sized ozonized poppy oil for infertility treatment.

Another purpose of the invention is to provide the use of solutions comprising nano or micron-sized ozonized oils for the pelvic inflammatory diseases.

Another purpose of the invention is to provide the use of solutions comprising nano or micron-sized ozonized oils for eye diseases. The solutions comprising nano or micron-sized ozonized oils can be used in eye infections, allergic eye diseases, autoimmune eye diseases, and similar eye diseases, preferably as eye drops or in different forms.

Another purpose of the invention is to provide the use of solutions comprising nano or micron-sized ozonized oils for the diseases relating to ears. The solutions comprising nano or micron-sized ozonized oils can be used in external, or middle ear infections and similar ear related diseases, preferably as ear drops or in different forms.

Another purpose of the invention is to provide the use of solutions comprising nano or micron-sized ozonized oils for the diseases relating to the nose. The solutions comprising nano or micron-sized ozonized oils can be used in sinusitis, colds, allergic rhinitis and similar nasal diseases, preferably as ear drops or in different forms.

Another purpose of the invention is to provide the use of solutions comprising nano or micron-sized ozonized oils preferably as a spray or in different forms in the treatment of flu.

Another purpose of the invention is to provide the use of solutions comprising nano or micron-sized ozonized oils preferably as a pastille or in different forms in the throat infections.

Another purpose of the invention is to provide the use of solutions comprising nano or micron-sized ozonized oils in the treatment of oral and periodontal diseases. The solutions comprising nano or micron-sized ozonized oils can be used in the treatment of diseases such as stomatitis, gingivitis. The solutions comprising nano or micron-sized ozonized oils can be injected into the cavities in tooth decays. The solutions comprising nano or micron-sized ozonized oils can be used together with a local anesthetic. The solutions comprising nano or micron-sized ozonized oils can be used in the treatment of dental abscess through injection. The solutions comprising nano or micron-sized ozonized oils can be used after tooth extraction and in the treatment of alveolitis.

Another purpose of the invention is to provide the use of solutions comprising nano or micron-sized ozonized oils preferably as a stomach-dissolving capsule or in different forms in the treatment of gastric diseases, and in the eradication of Helicobacter pylori.

Another purpose of the invention is to provide the use of solutions comprising nano or micron-sized ozonized oils that have a generalized systemic effect and solved in the intestine as capsules or in different forms.

Another purpose of the invention is to provide the use of solutions comprising nano or micron-sized ozonized oils in the treatment of hemorrhoids, fistulas, fissures, anal abscesses, and similar diseases as suppositories or in different forms.

Another purpose of the invention is to provide the use of solutions comprising nano or micron-sized ozonized oils in the treatment of colitis ulcerous, Crohn's diseases or in order to achieve the absorption of ozone from the intestine in general as suppositories or in different forms.

Another purpose of the invention is to provide the use of solutions comprising nano or micron-sized ozonized oils for rectal washing and lavatory.

Another purpose of the invention is to provide the use of solutions comprising nano or micron-sized ozonized oils as an intraperitoneal injection for peritonitis.

Another purpose of the invention is to provide the use of dilute solutions comprising nano or micron-sized ozonized oils for parenteral administration.

Another purpose of the invention is to increase the skin absorption of the products by using solutions comprising nano or micron-sized ozonized oils in cosmetic products.

Another purpose of the invention is to provide the use of solutions comprising nano or micron-sized ozonized oils easily in the desired density in sensitive tissues such as intestine, bladder, vaginal tissue, rectal tissue, mouth and nasal mucosa, eye and ear.

Another purpose of the invention is to enable the preparation of mixtures with high water content at the desired dosage in intra-articular injections for medical use.

Another purpose of the invention is to provide efficient use of drugs that require slow absorption and long release by providing the solutions comprising nano or micron-sized ozonized oils of the invention.

Another purpose of the invention is to provide the use of solutions comprising nano or micron-sized ozonized oils in veterinary medicine to replace antibiotics to prevent infections.

Another purpose of the invention is to provide the use of solutions by mixing it with feed and drinking water to improve the development of animal health.

Another purpose of the invention is to provide the use of solutions comprising nano or micron-sized ozonized oils as a regional injection and for administration into the cavity in mastitis in veterinary medicine.

Another purpose of the invention is to provide the use of solutions comprising nano or micron-sized ozonized oils as vaginal suppositories or in different forms in cases of vaginal infections in veterinary medicine.

Another purpose of the invention is to provide the use of solutions comprising nano or micron-sized ozonized oils for the treatment of abscess or regional infections in veterinary medicine.

Another purpose of the invention is to provide the use of solutions comprising nano or micron-sized ozonized oils for joint injuries or tendinitis in veterinary medicine.

Another purpose of the invention is to prepare products using solutions comprising nano or micron-sized ozonized oils in the cosmetics, food, veterinary medicine, medicine, and agricultural medicine, etc. at adjustable doses to be used in application areas.

Another purpose of the invention is to provide the production of disposable wet wipes from the solutions comprising nano or micron-sized ozonized oils.

Another purpose of the invention is to provide the production of disposable packaging solutions from the solutions comprising nano or micron-sized ozonized oils.

Another purpose of the invention is to provide the production of disposable hygienic vaginal mixtures from the solutions comprising nano or micron-sized ozonized oils.

Another purpose of the invention is to provide the use of solutions comprising nano or micron-sized ozonized oils in the form of gargle (mouthwash).

Another purpose of the invention is to provide the use of solutions comprising nano or micron-sized ozonized oils for the elaboration of cosmetic preparations, in skin care and treatment.

Another purpose of the invention is to prepare disposable packages to ensure that solutions comprising ozonized oil in nano or micron sizes are used by mixing into drinks.

Another purpose of the invention is to prepare or produce drinks with solutions comprising nano or micron-sized ozonized oils.

Another purpose of the invention is to provide the use of solutions comprising nano or micron-sized ozonized oils in combination with cleaning supplies.

Another purpose of the invention is to provide a brighter appearance to the fruits and vegetables and prolong their shelf life by spraying solutions comprising nano or micron-sized ozonized oils on them.

Another purpose of the invention is to provide a prolonged shelf life of the food of animal origin such as meat, fish etc. by spraying solutions comprising nano or micron-sized ozonized oils on them.

A further purpose of the invention is to provide the use of solutions comprising nano or micron-sized ozonized oil in the spraying of plants or in the organic farming applications.

Another purpose of the invention is to provide the use of solutions comprising nano or micron-sized ozonized oils in chicken breeding.

Another purpose of the invention is to provide an effective solution for the elimination of endo and ectoparasites in the animals, humans, and plants.

Another purpose of the invention is to provide a solution for the destruction and degradation of pesticides and insecticides on fruits and vegetables.

Another purpose of the invention is to extend the shelf life of fruit and vegetable products by using an apparatus comprising the ozonized solution to the stems in order to increase the durability period in fruits and vegetables.

Another purpose of the invention is the use of the ozonized solution alone or in combination with auxiliary products such as thymol in bees to treat nosema disease, varroa, American rot disease, European rot disease, lime disease, as a hive disinfectant and to improve bee health.

A further purpose of the invention to prevent the production of dried apricot mites on the apricot and the resulting contamination by using ozone solutions by dipping and spraying methods.

A further purpose of the invention is to provide a solution with a repellant effect, which is found in studies to inhibit aphids, caterpillars and other insects from eating of leaves and expel them from the environment at the same time with the use of various doses.

Another purpose of the invention is the use of ozone solutions in the transport of liquid foods such as milk and to ensure the long-term durability of them without boiling.

Another purpose of the invention is to provide a solution with a repellant effect which acts both as a disinfectant and as a shaft louse and flea removing agent in poultry farms.

Another purpose of the invention is to provide a solution with an insect repellent effect in dry pulses.

Another purpose of the invention is the use of it to keep seeds in the soil and to prevent the formation of seed infections.

Another purpose of the invention is to provide a solution that prevents both infections and contributes to plant growth by applying them to the roots in greenhouse cultivation and soilless agriculture by mixing with water.

Another purpose of the invention is to provide a solution that allows the removal of parasites such as louse and fleas in pets in the form of sprays or shampoo.

Another purpose of the invention is to provide the ease of transport by removing the water by lyophilizing the ozonized solution. It can be used by re-dilution in this form. Another purpose of the invention is to provide the use of it in space technology to produce plants in anaerobic environments. It provides benefits for plant development in the anaerobic environment in the form of a solution.

Another purpose of the invention is to provide an ozonized solution comprising saline water, which enhances the effect in products such as nasal drops, ear drops, eye drops.

Another purpose of the invention is to combine all vitamins and other anti-hair loss agents with ozonized solutions in order to increase efficacy in combinations preventing hair loss.

The use of fat-soluble vitamins in nanoparticles is a new form of use.

A further purpose of the invention is to provide a new method of use by injecting ozonized solutions with all vitamin combinations into the tissue in form of nanoparticles and to provide an enhancing effect to the tissue healing properties.

Another purpose of the invention is to provide ease of use in food supplement, toothpaste, mouthwash, animal food supplements by the formation of ozonized solutions with sugared water or sweetener.

A further object of the invention is the use of ozonized solutions in combination with oil or petroleum products as a fuel additive to improve engine performance.

Another purpose of the invention is to provide the fabric with antibacterial properties and to disinfect the fabric as a result of using solutions with ozone in the textile with washing, dipping, spraying methods or immersing the rope in solutions with ozone before the knitting. In order to enhance the desired effect, the density of the solutions and ozone dosage can be increased.

Another purpose of the invention is to provide the digestive system preparations formed by the intensive doses of ozonized solutions in 3 different forms, namely, stomach-soluble capsules, small intestine-soluble capsules, and lower intestine-soluble capsules. It provides different advantages in each region.

Another purpose of the invention is to use it as an effective treatment method for oral infections such as stomatitis, gingivitis by applying directly to the gums or in the form of toothpaste. The solutions comprising nano or micron-sized ozonized oils can be used in oral care products in various forms such as gargle (mouthwash), toothpaste additive.

A further purpose of the invention is to provide an effect to enhance fracture union by injection of the ozonized solution into and around the fracture hematoma. The use of it in combination with vitamin K, vitamin D, calcium, etc. accelerate the fracture union.

Another purpose of the invention is the use of it in combination with hyaluronic acid in the intra-articular injection to benefit from its effect of increasing cartilage matrix. It is known that better results are obtained with the ozone treatment and hyaluronic acid combinations. In the studies of the invention, ozonide solutions in water are mixed at the ratio of 1-30% and the use of the effective solution in the joint with the addition of 1-2% hyaluronic acid increases the effect on cartilage healing.

Another purpose of the invention is to present an ozone solution comprising poppy oil.

The use of the poppy oil injection through the hepatic artery in liver tumors constitutes effective treatment. Poppy oil is ozonized in nano or micron sizes, given in the same way, so that, its entry into the cancer cell is facilitated and its effect can be increased.

Another purpose of the invention is to present an ozone solution comprising St. John's Wort oil (the extract of Hypericum perforatum obtained with olive oil). The effectiveness of ozone oil made with Hypericum perforatum (St Johns-wort) is known in the treatment of hemorrhoids. The solutions comprising nano or micron-sized ozonized oil of Hypericum perforatum are used in the treatment of hemorrhoids in the form of suppositories.

In order to fulfill the purposes listed above, the invention relates to the solutions comprising nano or micron-sized ozonized oils in designated ozone doses, comprising oil, ozone gas, water, and at least one emulsifier.

The structural and characteristic features and all advantages of the invention outlined in the drawings below and in the detailed description made by referring these figures will be understood clearly, therefore the evaluation should be made by taking these figures and detailed explanation into consideration.

### DETAILED DESCRIPTION OF THE INVENTION

In this detailed description, the preferred structures of the solutions comprising nano or micron-sized ozonized oil, their production methods, and their usage areas are explained only for better understanding of the subject.

Known uses of ozonized oil or ozonides are defined as for wound treatment, for skin diseases, for cosmetics, or for uses in the digestive system. The application of ozone by parenteral means, the application of ozone by local injection, the application of rectal ozone, major therapy, minor therapy, regional ozone applications with bags and hearts are the main ozone application methods. However, to perform these applications, there is a need for a clinic with an ozonizer or a hospital. The targeted treatment method aims to ensure the widespread use of ozone by making it available in pharmacies with portable vials or ampoules. In addition, it is to ensure the use of it in veterinary medicine, food industry, nutritional solutions, chemical industry, agriculture and to turn it into salable products in markets.

In the process of preparing solutions comprising ozonized oil, the oils that are known as the best movers of ozone and as keeping it stable for a long time are ozonized. The structures called ozonide are formed with the ozonization of oils. Basically, it is known that all oils can be ozonized. The oils used in solutions comprising ozonized oil of the invention are divided into two as injectable oils and non-injectable oils. Oils of vegetable origin used for injection purposes such as soybean oil, sesame oil, palm oil, poppy oil, soy lecithin, cholesterol, b-sterol, triglyceride, olive oil, fish oil, sunflower oil, castor oil, saffron oil (safflower), coconut oil, triglyceride derivatives, tributyrin, tricaprin, tricaprylin, and vegetable and animal fats for the purpose of feeding from the vascular way, for a person of 70 kg, adding up to 175 grams of fat emulsion to dietary solutions is a widespread method. Ozonides formed by the ozone bubbles formed in the oil as a result of ozonization of all injectable oils such as paraffin (mineral oil), ethyl oleate, methyl oleate used in the chemical industry are used individually or as a combination in certain doses in order to get rid of the unique side effects of each oil.

Mixtures of solutions obtained from the ozonization of the non-injectable oils are used generally as a cream, ointment etc. in semi-solid semi-liquid form for cosmetic purposes and wound treatment. Injectable solutions cannot be prepared with ozonized oils and ozonides obtained by the current techniques. When the ozonized oils and ozonides obtained by the current techniques are used in injections, due to their burning effects, they damage the tissues and show low absorption. All the oils used are subject to different rates of ozonization depending on their content.

Injectable oils are of vegetable and chemical origin. Oils of vegetable origin display exothermic reaction and give heat to the outside during the ozonization process. Therefore, during the ozonization of oils of vegetable origin, a cooling process should be applied. Long term ozonization of oils of vegetable origin causes their solidification. In this case, it is not possible to inject them. In the ozonization process of oils of chemical origin like petroleum derivatives such as paraffin, since they do not display exothermic reactions, their density remains almost stable. However, the injection of these oils of petroleum derivatives with intense ozone damage the tissues. In addition, oils and ozonides in the form of large particles make them difficult to absorb.

In its simplest form, the solutions comprising ozonized oil comprises oil, ozone gas, distilled water, and emulsifier. In the studies conducted within the scope of the invention, it was observed that the water used in solution making can be prepared with saline water, sugared water, mineral water, deionized water, demineralized water, spring water, saline solution or physiological saline solution according to the area of use instead of distilled water and this has perfected the invention. For example, the solutions prepared for the nasal drops that contain salt water or the ones used in the beekeeping industry that contain sugared water increased the efficacy. In preferred embodiments of the invention, solutions comprising ozonized oil may comprise additional substances such as water and/or oil-soluble vitamins, minerals, hyaluronic acid, plant extracts or active ingredients, essential oils, or thymol, in order to increase efficacy relative to the area of use. In studies conducted, it has become clear that the property that determines the efficacy of the solution of the invention is that the amount of ozone gas in its content can be measured and standardized in mg. In the studies, it was revealed that the doses that were perfected by the invention were 10-100 000 mg/L (ppm) in ozone gas content. The doses determined in the solutions were ascertained to remain stable for 1 year at room temperature and it is predicted that in cold storage, for example at +4° C., it will extend over 10 years. The content and usable quantity by weight/volume in the invention within these designated doses are indicated in Table 1. All samples which can be formed within Table 1 are within the scope of the invention. The said solutions are the solutions comprising ozonized oil with a particle size range of 1 nanometer to 400 micrometers. Due to these properties, these solutions are suitable for injection.

TABLE
Contents and amounts of use

| Contents | Usable Quantity (%) |
|---|---|
| Oil | 1-70 |
| Ozone gas | 0.001-10 |
| Water | 10-97.9 |
| Emulsifier | 1-50 |

In preferred embodiments of the invention, the oils that are used for injection and have been studied in terms of their damage to the body, are ozonized. The vegetable originated ones of these oils perform the exothermic reaction in the ozonization stage and their density increases. However, hydrocarbon derivatives (oils of chemical origin) do not form an exothermic reaction, and their structure and density do not change.

Ozone generators are used as sources of ozone in the process of ozonization. Ozonization process is carried out with the use of ozone/oxygen mixture obtained from these devices. In long-term ozone processes, when the oils before and after processing are weighed with precision balance, the density of ozone in vegetable oils is observed at 160 mg/gr, and in hydrocarbon oils at 120 mg/gr. This constitutes a good and reliable measurement method for measuring the amount of ozone in the oil. In injection applications, calculations are also made on micrograms. For example, in a session of major therapy, 100 ml (cc) ozone in a density of 30 micrograms is applied, so that a total of 3 mg of ozone is given mixed with oxygen. The approximately same doses are used in local applications. It is concluded that intramuscular or subcutaneous injection of ozonized oil or ozonide at the same doses will have the same effect as ozone release. Intravenous administration of ozonized oils or ozonides has been studied in the experiments on rats and bleeding has been observed in alveoli. However, in the studies made with the solutions comprising nano or micron-sized ozonized oil, rats were given a very high dose of ozonized oil or ozonide by subcutaneous injection and the ozone application of 1 mg/gr subcutaneous ozone for 3 days in a row, it is revealed that these did not cause any death, but rather an increase in activity of rats. In the experiments conducted with rats, in the pathological examinations and laboratory tests 0.5 ml of solutions comprising 10 ppm-50 ppm-100 ppm-500 ppm-1000 ppm-5000 ppm ozone; which means comprising 10 mg-50 mg-100 mg-500 mg-1000 mg-5000 mg ozone per 1 liter; were subcutaneously injected per week and no harmful situation was observed. In addition, when hair follicles are counted, an increase in the hair in the injection area was detected. Studies have shown that low-dose ozone solutions have a slight cytotoxic effect on tissue and that accordingly cause the stimulation of growth factors with the minimal damage on tissue and tissue recovery has begun following 24-48 hours of degeneration. While tissue damage from 10 ppm to 500 ppm is minimal, with the increased doses also the damage increases, yet the amount of oxygen released increases and enhances the anti-bacterial effect and anti-inflammatory effects. It provides ease of use to be applied with local anesthetics in order to avoid the pain and feeling of burn that occurs during the application. These doses were applied as 0.5 ml in rats. The same dose will show the same local effect in humans. No significant side effects were observed in the studies. The pathological examinations reveal that it did not cause any damage to the liver, kidney or lung tissues. This situation showed that 3-4 ml of ozone solution can lead to good results and be used safely in intra-articular applications in combination with local anesthetics. Considering the rats are 250 grams in weight, this dose is equivalent to 35-70 ml ozone for a 70 kg person compared to the treatment dose of 4 ml ozone applied to humans. This reveals the fact that the safety range is very high and that ozone doses can actually be used at much higher rates. Considering the regional improvements, it is thought that the safety interval will be very high as the application of 3-4 ml ozone solution to 70 kg human will be effective. The ozonides being a dense oil solution becomes an advantage. Subcutaneous and intramuscular applications of dense oils provide slow release of the drugs in the oil and form long-release drugs. The injection of bupivacaine in oil provides a local effect for 72 hours. When combined with ozonized oil or ozonide, it will benefit both from the long-term antioxidant effect of high dose ozone due to regional long-term ozone release and also can reduce regional injection pain as well. Especially to eliminate the infection and the postoperative pain and to ensure the anti-inflammatory effect, a regional injection at the end of the operation accelerates wound healing as well as reducing regional pain and preventing infection from occurring. If it is used together with analgesic local anesthetics, the postoperative pain is reduced. The same effect applies to intra-articular ozone applications.

Considering that there is about 100 mg ozone in 1 ml ozonized oil or ozonide, the ozone amount in 0.1 ml oil is about 1 mg. Under normal circumstances, the ozone/oxygen mixture made with ozonizer is given into the joint as 20 microgram of ozone in 20 ml (cc). 2.5 times the dose of 400 micrograms of ozone is provided with such a small amount of oil. It is also known that together with the ozone in ozonized oils or ozonides is measured in grams, and its interaction with the oils, it ensures that the oils become saturated and its structure is changed. It is said that the mechanism of action oxidizes the area in which it is applied, causing damage in the tissue and therefore activates the repair mechanism of the body. It is known that as a result of ozonization of oils, ozone is bound to the carbons as O2 and O— and both are transferred to the tissue. In high doses, it oxidizes the tissue and creates tissue damage. In low doses, it is released slowly from the oils and thus oxygen is released into the tissue as well as free oxygen. This enables tissue healing and provides bounding of free radicals. The healing mechanism that is seen in wound treatment and the reactions seen in skin creams reveals the increase in oxygen transport to the tissues. When applied on the skin, the amount of oxygen absorbed into the subcutaneous tissues is increased and the color of skin is enlightened. It is observed that both the wound area and the repair mechanism is activated by oxidation and the free oxygen in the ozonized oil or ozonide is transferred to the tissue which resulted in an increase in the oxygenation of the tissue. Its antibacterial, antiviral, antifungal properties and anti-inflammatory and analgesic effects are other wound healing effects. It is thought that these effects will also be activated in regional injection applications just as they are activated in epidermal applications. In studies conducted on rats, it is seen that subcutaneous injections of a high dose such as 1 mg/gr do not cause the death of rats, but cause skin necrosis together with regional hair loss and scar formation. Considering that this injection dose is 70000 times more than the regional injection dose applied to human, it means to apply 70 grams of ozone injection to a person of 70 kg on the same area will be done 3 days in a row. With the use of these doses, rats were monitored for a month and no deaths occurred. The density of ozonized oils or ozonides can make injection difficult and cause regional redness and necrosis. For this reason, the oil emulsions in the prepared water is another form of application. Ozonized oil or ozonides are dissolved in water using many various methods (ie. with ultrasonic oil digesters, nanotechnological methods, etc.) to make solutions comprising ozonized oils or ozonides. In microscopic examination, ozonized oil or ozonides having a nano or micron particle sizes are suspended in water in a wide range from 1 nanometer to 400 microns. These solutions can optionally be prepared at concentrations ranging from 1% to 90%.

The emulsifiers used to form oil emulsions (solutions) have been successfully used for years to form intravenously nutritional solutions. The emulsifiers such as the lecithin formed from egg yolk, lysophospholipid (12), polyethylene glycol (PEG), phosphatidylethanolamine (PEG-PE), pluronic, Polysorbate 20, Polysorbate 80 are applied and currently marketed in the products. These are safely used emulsifiers. While one or more of these agents are used in human and animal health, emulsifiers for general use can also be preferred in other industries.

The formation of oil particles in nano or micron sizes can be done with various methods. Solutions comprising ozonized oil in nano or micron sizes of the invention are preferably made in two ways. In either way, preferably, the oil or oil mixtures that the application will be made are ozonized first, then nano or micron particle creation and emulsion process can be done. However, nano or micron particles can be created first and then ozonization can be applied. In this case, the particle size increases.

The production method of solutions comprising ozonized oil in nano or micron sizes of the invention comprises following process steps;
A. Ozonization of selected oils until they absorb the specified amount of ozone gas,
B. Mixing of water with at least one emulsifier,
C. Addition of ozonized oils to the mixture comprising water and at least one emulsifier and blending of them in the mixer with the cutting blades,
D. Spray cooling of the solution.
E. Reduction of particle sizes of the solution comprising ozonized oil down to 1 nanometer using ultrasonic cutting method.

The oils used in the process step A are preferably injectable oils. In preferred embodiments of the invention, oils of vegetable and animal origin such as soybean oil, sesame oil, palm oil, poppy oil, St. John's Wort oil, soy lecithin, cholesterol, b-sterol, triglyceride, olive oil, fish oil, sunflower oil, castor oil, saffron oil (safflower), coconut oil, triglyceride derivatives, tributyrin, tricaproin, tricaprylin, or oils of chemical origin (hydrocarbon derivatives) such as paraffin (mineral oil), ethyl oleate, methyl oleate are used. A single type of oil may be used in the preparation of the solutions of the invention, as well as an oil mixture comprising different oils. While the most preferred oil for the preparation of the solution of the invention is sunflower oil, according to the application area, olive oil, soybean oil, poppy oil or St. John's Wort oil can be used. In the process step A, oils are subject to ozonization for preferably 10 days. The duration of the ozonation process depends on the type and quality of the oil used and the usage area. Ozone generators are used as sources of ozone in the process of ozonization. Ozonization process is carried out with the use of ozone/oxygen mixture obtained from these devices. The amount of ozone in the oil can be determined by measuring the mass of the oil that is subjected to the ozonization process before and after step A. Precision scales are used for the measurement process. In this way, the amount of ozone in the oil is determined in milligrams.

Ozonization process varies according to the intensity of the ozonizer. For example, oil is measured in grams with an industrial ozone generator having an ozone output of 100 grams per hour. After 3-4 hours 10-15% of ozonized oil is obtained. In this way, 10 kg of oil becomes 11-11.5 kg in weight. This varies depending on the type of oil. If the volume of the ozonizer is reduced and the volume of oil is increased, the ozonization process time is extended. Usually over 10%, the ozonization process takes a long time.

Since ozone devices are not standard, the ozone and oxygen rates they contain cannot be fully determined. For example, not only the amount of ozone in the gas emitted from the ozone generating machine with a source comprising 90% oxygen is less, but also other foreign gas quantities mix into the oil. In this case, when we accept the oil as 15% ozonized, we calculate it wrong. When the oils are saturated only with oxygen, they are aggravated by oxygen up to 6%. At the same time, in the process performed by closing the oxygen supply, the oil does not receive any ozone from the air, but because the oxygen content is very low, nitrogen and other gases in the air are also loaded, and an ozonized oil with a much-altered standard is obtained. Because this is used to create injection and ozone solutions in very small doses are required, the importance of these doses as actual doses becomes evident. The ozonization of the ozone solution to be made suggests that the oxygen production of the ozone device requires a significant standardization. It is important that if the dosage of ozone solution for injection is adjusted with one device, then the production should be done at the same standards and experimental studies should be conducted with the same devices. At the same time, it is important to reduce the metal load with devices such as ionizers or magnets, or to use glass-covered corona discharge devices, as the mixing of electron breaks in the use of metal apparatus corona discharge devices with ozone gas can also cause a problem.

Ozone bearing capacity of oil decreases over time. In such a case, the oil is saturated in a few days and became frozen while the ozonization process continues. Saturated oil may be dissolved again and then subjected to ozonization. In one embodiment of the invention, this process is repeated several times and 1000 g olive oil increase by 160 g and becomes 1160 g olive oil with ozone or ozonide in 8-10 days. This situation is usually valid for all vegetable oils. Since hydrocarbon oils such as paraffin do not freeze, heating is not required. In an embodiment of the invention, 1000 g of paraffin becomes 1100 g when it is subjected to 3 days of ozonization. In such oils, ozonization as much as olive oil is not formed. The high ozone process makes the oil more acidic. In this step, the amount of ozone in the oil is calculated in grams and the amount of ozone to be added according to the amount of oil added to the mixture is determined. In other words, if 10% of ozonization is made in 10 grams of oil, 1 g of ozone will be present and 1000 ppm of ozone will be found if this amount is to be completed in a liter of water. If ozonization is made by 15%, the rate will be 1500 ppm. This is important to produce pharmaceutical formulations. In the applications of the current technique, oils with ozone are emulsified but the amount of ozone gas in the oil is not specified. In order to determine the doses that perfected the invention, studies are carried out with the calculation of ozone in ozonized oil in all steps of product creation.

To give a sensitive example, when 50 apricot mites were placed on dried apricots and left in Petri box, it was seen that mites formed colonies. These mites can be killed by ozone gas, but a few days later, new apricot mites emerge from the eggs and reproduce and then form colonies. This situation causes apricots to be a harmful food on which bacteria and fungus reproduced that causes allergic diseases when consumed together with the feces of mite. In our study, 500 ppm, 1000 ppm, and 2000 ppm ozone solutions were used in the solution, and it is seen that the apricot mites were eliminated in 60% in 48 hours at 1000 ppm and eliminated in 90% at 2000 ppm. The efficacy extended up to 14 days and also the mites hatching from the eggs were destroyed. At higher doses, the efficacy increases, but the bitter taste of ozone is felt in apricot. This shows how important the dose is. Even though the oil is ozonized by 10% or 5% or 15%, it should be in a dose comprising 2000 ppm (mg/liter) ozone into the solution. In other words, if ozonized oil comprising 5% ozone will be used in the solution, then 20 g of oil will be used per liter, if 10% of ozonized oil will be used, then 10 g of oil will be used and if 15% of ozonized oil will be used, then 7 g of oil will be used per liter.

In the process step B, the temperature of the mixture comprising water and at least one emulsifier is 10-15° C. above the melting temperature of the oil depending on the type of oil used. In a preferred embodiment of the invention, distilled water is heated up to 70° C. and at least one emulsifier is added at the process step B. At the end of the process step B, the temperature of the distilled water and emulsifier mixture is 70° C. The water and emulsifier are mixed preferably at 11000 rpm for 5 min in process step B. The emulsifiers such as the lecithin formed from egg yolk, glycerin, lysophospholipid (12), polyethylene glycol (PEG), phosphatidylethanolamine (PEG-PE), pluronic, Polysorbate 20, Polysorbate 80 or different substances can be used as emulsifiers. In a preferred embodiment of the invention, Polysorbate 80 is used as an emulsifier. Due to the emulsifier, water and oil are mixed within each other.

In the process step C, the temperature of the mixture comprising water, at least one emulsifier and ozonized oil is 10-15° C. above the melting temperature of the oil depending on the type of oil used. In a preferred embodiment of the invention, ozonized oil (or ozonide) at 70° C. is added to the distilled water and emulsifier mixture at 70° C. in the process step C. The mixture is blended at high speed within the mixer having the cutting blades. In a preferred embodiment of the invention, mixing is performed using speeds between 11000 to 13600 rpm. Thus solutions comprising disintegrated ozonized oil (or ozonide) having particle sizes of 1 nanometer to 400 micrometers are prepared.

An alternative method is the use of high-speed motors to create a nanoparticle. The production made with these devices preferably at 36000 rpm speed does not require heat treatment and then the cooling process will not be necessary. The use of these devices allows both the degradation of oils in the medical field, the cosmetic industry and the chemistry, as well as the separation of extracts and powders, making the solution easier to obtain and obtaining nanoparticle solutions. The use of this type of device is important in these applications as it is feared that the heat treatment may reduce the amount of ozone. This type of cold particle formation process should be standardized in the drugs with the ozone solution. While the oil can stand at +4 degrees for years, the ozone content is lost at +20 degrees in 1-2 years, at +40 degrees for a few months, and at +70 degrees within hours. In this case, the drug dose will not be able to calculate accurately and precisely. The process step D is not used in this method since the heating will not be applied.

In the process step D, solutions comprising water, at least one emulsifier and ozonized oil (or ozonide) are cooled via the spraying process. In this process step, the resulting solution is allowed to cool suddenly by spraying to a cold area. Keeping the disintegrated oil warm for long time results in particle growth. The cooling process is applied to prevent this. Preferably, cold homogenization devices are used for the cooling process. In a preferred embodiment of the invention, solutions comprising ozonized oil in nano or micron sizes can be cooled to temperatures down to 4° C. or lower. Although there is no specific temperature range for cooling, high temperatures can cause particle growth.

In the process step E, the particle sizes of the solution comprising ozonized oil are reduced down to 1 nanometer using ultrasonic cutting methods. Ultrasonic cutting methods are carried out with pieces of equipment such as ultrasonic splitter or reactor etc. In a preferred embodiment of the invention, solutions comprising ozonized oil with particle sizes within the range of 10-50 nanometers can be used. The solutions of the invention include ozonized oil in the range of 1 nanometer to 400 microns.

In the development studies carried out within the scope of the invention, it is found that nanotechnological methods such as high-pressure homogenizers, micro-fluidization, ultrasonic emulsification, electrolysis or zeta potential can be used as an alternative to form micro or nanoparticles in the production of the ozonized solution.

The oil to be used for the preparation of the solutions of the invention is weighed primarily through the precision scale and the result is recorded. The oil is subject to the ozonization with the ozone/oxygen mixture obtained from the ozone generator. Following the ozonization process, the measurement is made again with the precision scale and the amount of ozone in the oil is obtained in milligrams. Then ozonized oil (or ozonide) at 70° C. is added to the mixture comprising distilled water and preferably polysorbate 80 at the same temperature and preferably mixed with the mixer comprising cutting blades at high speed such as 11000 to 13600 rpm. The resulting solution consists of ozonized oil (or ozonide) particles at sizes between 1 nanometer to 400 micrometers. This solution is cooled down by spraying to a cold area. Finally, the solution to be prepared is packed according to the desired form, content and quantity.

If hot homogenization is to be carried out in the method of production described above, after mixing the water with polysorbate 80 in a ratio of 2-5% at 70° C., the ozonization of the stable nano or micron-sized particles obtained by the rapid spraying of the nano- or micron-sized particles formed by mixing above 10° C. of the melting point of the oil with a mixer of cutting blades at a speed of 11000-13600 rpm is another alternative method. However, the hot environment may cause the ozonides to dissolve and may reduce the ozone levels in the oil. It is possible to create nanoparticles by cold homogenization method to get rid of the effects of hot homogenization. Particle size can be adjusted according to the application area. The disintegration of nano or micro particles formed with hot or cold homogenization again by ultrasonic means allows the formation of smaller particles.

Solutions comprising ozonized oil in nano or micron sizes of the invention can be produced by ultrasonic methods. Ultrasonic oil shredders are used in these methods. Heat treatment is not used in the formation of particles in nano or micron size by the disintegration of the oils with ultrasonic methods. During the application, cooling can be applied. There is no need to use emulsifiers in this method. Because particles smaller than 1 micron can be formed. As the application time increases, the particle size is reduced. With this method, oil particles can be reduced from 1 nanometer to 400 micrometers.

As a third production method, it is possible to create solutions comprising ozonized oil in nano or micron sizes by the potential method of Zeta. Electrolysis is used in this method. Application of 25 mV is acceptable for stable solutions. The creation of ozonized oils or ozonides in nano or micro sizes enables the adjustment of dosage and the use of ozone more safely by creating dilute solutions. Problems such as absorption disorders resulting from the injection of oils, the remaining oil in the tissue and forming an abscess infection are eliminated due to the nano or micro particles. For example, it is possible to prepare solutions from 1 ml to 5 ml comprising 1 mg of ozone. In this way, it is easier to inject into the areas such as the joints. At the same time, the duration of stay in the tissue according to the intensity can be calculated and other drugs can be injected into the tissue together with ozone to provide long-acting. This is best understood by local anesthetics. Lidocaine, which is normally effective for up to 2 hours in the injection zone, increases the duration of the effect compared to the injection of the oil. Within this period, the regional interactions of other drugs and the release time of systemic drugs can be calculated. It is especially important that the application of ozonized oil or ozonide to subcutaneous tissues should not be in intensive doses and should be in diluted solutions. In order not to have subcutaneous tissue necrosis, solutions in water comprising 1-10% ozonized oil or ozone are preferred. In the applications to deep tissue, muscle and adipose tissues, more intense doses should be taken. However, given that a regional injection dose of ozone is 3-5 mg, the application can be performed easily with 1 ml of the solution having 5% ozone. In wound treatments, creams of 10-15 gr with ozonized oil or ozonide comprising 160 mg/gr ozone are regionally applied. This intensive ozone dose is more effective in wound treatment, and that the low-dose ozone-comprising creams did not cure the wound quickly was observed in studies. Considering all these, it is thought that a higher dose of ozone should be given in regional injections yet ensuring that there are dilute doses. For this purpose, the solution of ozonized oil or ozonides in the water is easy to apply at the rates that vary according to the region in dosage adjustment. It also facilitates regional absorption through the creation of nano or micron-sized particles, thereby allowing it to be absorbed from the region without causing tissue necrosis and allergic reactions.

Diluted ozone solutions can be prepared by adjusting the ozone dose in both ozonized oil and ozonide and by adjusting the density in solution in water. It is thought that there is a large amount of ozone in the low amount of oil in the injection of concentrated ozone or ozonides. The less oil is used in injection, the less the side effect of the oil experienced. For this reason, solutions of the oils with high-ozone in water are advantageous.

Dosage adjustment of the solutions comprising disintegrated ozonized oil in nano or micron sizes can be made and dilute solutions can be prepared. Thus, ozone is used more safely. The solutions comprising ozonized oil in prepared nano or micron sizes are prepared in doses varying according to the usage areas and the apparatus used.

If the solutions of the invention are to be used in the suppository form, the formed mixture is cooled down by adding the substances such as Witepsol.

When the solutions comprising ozonized oils of the invention are combined with drugs such as insulin or local anesthetic drugs, they prolong the release and action times of these drugs and facilitate their absorption.

Since the injection of solutions comprising ozonized oil of the invention will be released slowly in regional infections, it increases oxygenation and reduces the use of antibiotics. It can be applied as an injection into the abscess and periphery of it. In order to speed up the postoperative wound healing, it can also be injected to the surgical region.

The intramuscular injection of the solution comprising ozonized oil of the invention mixed with 5-10 ml (cc) of venous blood is equivalent to the treatment method known as minor therapy.

The said solutions comprising ozonized oil can be used in all external forms and mucosal forms together with silver nanoparticles. The solutions comprising nano or micron-sized ozonized oil of the invention can be used as combined with silver or similar other metals that shows oligodynamic effects.

In products for cosmetic purposes, solutions comprising ozonized oil can be combined with hyaluronic acid and fat-soluble vitamins.

The Use of Solutions Comprising Ozonized Oil in Orthopedics

As known, the cause of regional pain is the decrease in tissue oxygenation due to muscle spasm and the increase in inflammation due to it. In regional ozone applications, ozone/oxygen gas is given to the pain zone as superficial and deep. 20 micrograms of ozone gas 40-50 ml (cc) is injected locally to provide an anti-inflammatory analgesic effect. At the same time due to the increase in tissue oxygenation, muscle relaxant effects are obtained. Ozone oil in nanoparticles or micron sizes dissolved in water can be injected regionally by subcutaneous and intracellular ozonized oil or ozonide application. It is known that paraffin oil has muscle relaxant properties in the injection. Ozone concentration used in regional applications is in doses of 1-2 (1000-2000 ppm) mg with ozonized oils comprising preferably 10% ozone, yet, since the dissolution of ozone in oils can be prolonged up to 72 hours, it can be raised up to 5-6 mg doses. This application can be done in the same area at most twice a week. Local anesthetics lidocaine or bupivacaine, anti-inflammatory agents may be added to the solution.

Ozone gas injection may cause short-term burning and itching. However, this effect is rarely seen in intramuscular injections. For this reason, ozonide solutions comprising local anesthetic should be preferred especially if superficial injection will be performed. The effect of ozone on intraarticular injections has been revealed extensively in recent years. It is known that it prevents loss of cartilage by increasing oxygenation of the joint cartilage and reduces pain due to the analgesic effect with its anti-inflammatory effect. There are many publications in the literature about this issue. Similarly, in our own research, it was observed that VAS scores decreased from 8.5 to 4 on average as a result of the application of 8 sessions of ozone and 2 sessions of PRP treatment on 187 knees of 100 patients. In these applications, 20 micrograms of ozone are given as 20 ml (cc). That is, 400 micrograms of ozone have been applied. For the same application, it can be used in doses of 1-2 (1000-2000 ppm) mg with ozonized oils comprising preferably 10% ozone, as it will take up to 72 hours for the dissolution of diluted water solutions comprising nano or micron-sized ozonized oil or ozonide. It can be applied at most twice a week. In addition, it can be combined with local anesthetic and anti-inflammatory agents. Vitamin E, which becomes popular in experimental studies in recent years, can be applied together with oil solutions and anti-inflammatory drugs. Hyaluronic acid is known to increase cartilage matrix in the intraarticular injection. It is known that better results are obtained with the ozone treatment and hyaluronic acid combinations. In the studies of the invention, ozonized oil or ozonide solutions in water are mixed at the ratio of 1-30% and the use of the effective solution in the joint with the addition of 1-2% hyaluronic acid increases the effect on cartilage healing.

Cervical disc and spinal disc hernias are one of the common health issues. In the studies we conduct on 66 patients diagnosed with a cervical disc hernia, the pain score is evaluated according to the VAS in 1 month, 4 months and 1 year after the regional injections of 8 sessions of ozone, 2 sessions of PRP. The pain score was 9.3 at the beginning, 6.9 in the first month, 4.8 in the 4th month and 3.6 at the end of the first year. It is also found in the literature that the regional injections of ozone are so effective. However, to perform these applications, there is a need for a clinic or a hospital where ozone treatment is offered. The dosages of solutions of ozonized oil or ozonides in water in regional applications are also applicable here. Depending on the size of the region, the ozonized oil or ozonide solution of 2-4 mg (2000-4000 ppm), which is made with ozonides comprising 10% ozone may be injected into paravertebral muscles and subcutaneous tissue to be able to benefit from anti-inflammatory and analgesic effects. However, with tissue oxygenation, dissolution of muscle spasm, and increased blood supply to the hernia, a long-acting treatment and shrinking of the hernia is provided.

In order to accelerate the fracture union, regional injections of vitamin D have been tried in the literature. Solutions of ozonized oils or ozonides in water increase regional oxygenation. For this reason, the injection of ozonized oils or ozonides into and around the fracture hematoma has an effect that increases the fracture union. The use of it in combination with vitamin K, vitamin D, calcium etc. accelerates the fracture union.

Ozone application is one of the most effective methods in wound treatment. Wound treatments usually fail, and the organ loss is experienced. Both giving the ozone to the area of the wound with a bag in the region, and the major therapy with regional injection are known methods of ozone treatment. However, since ozone therapy is not very widespread and requires the ozonizer, it has a limited area of use. The regional injection of solutions of ozonized oils or ozonides in water and the use of intensive doses of oils in combination are effective in wound healing. It plays an effective role in wound treatment due to the use of 4-5 mg ozonized oil or ozonide solution in water as a regional injection and the superficial (epidermal) use of 160 mg ozonized oil or ozonide. The solutions of the invention can be used in wound treatment with silver nanoparticles.

Ozone therapy plays an effective role in the treatment of plantar fasciitis, tennis elbow, tendinitis and in the elimination of regional pain. Application of ozonized oil or ozonide solutions of 1-2 mg (1000-2000 ppm) in water prepared with ozonized oil comprising preferably 10% ozone to the area is an effective treatment method.

The Use of Solutions Comprising Ozonized Oil in Urology

Due to the disinfectant effect of the lubricants comprising chlorhexidine and lidocaine used in catheter applications are preferred to prevent the penetration of bacteria into the probe. The solutions of ozonized oil or ozonides in water are used as a true lubricant for both its nontoxic features and oil content. It is a highly effective lubricant when it is applied together with local anesthetic due to its long-term release of ozone in the oil and the longer lasting effect of local anesthetic release. Silicone probes should be preferred due to the negative interaction of ozone with plastic.

The use of solutions of ozonized oil or ozonides with anti-inflammatory, analgesic, antibacterial effects in the treatment of cystitis and urethritis, even with ureteral catheter applications has a positive impact on tissue healing in this region. In the treatment of cystitis, the solutions of ozonized oil or ozonides can be applied daily into the bladder with the probe. Intake of plenty of fluids is recommended. The intravesical and urethral contact with the ozonide solution accelerates the recovery via antibacterial and anti-inflammatory effects. Treatment is provided with the use of solutions of ozonized oil or ozonides alone or in combination with antibiotics.

The Use of Solutions Comprising Ozonized Oil in Gynecological Diseases

Genital area infections in women are viral, bacterial and fungal infections and are common and contagious diseases. The use of ozonized oil or ozonide can be an effective method for the cleansing of the vulva, vagina, and perineum. The use of disposable disinfectant wipes in order to facilitate disinfection is a practical method to avoid infections and unpleasant odors.

In order to prevent transmission of infections before or after sexual intercourse, the preparation of solutions of ozonized oil or ozonides in disposable packages which have both lubricating and disinfectant properties provides ease of use. Spraying of the disposable product into the vagina provides protection from the infections. The solutions of the invention can be used in the form of a vaginal ovule or in different forms.

The use of poppy oil in gynecological diseases and infertility treatment has been known for centuries. The use of poppy oil as an intravenous washing solution can be an effective method for the treatment of infertility because of the anti-inflammatory effect of it in the tubal and uterus, the canals will remain open and the negative effects of the autoimmune system will be eliminated.

In the treatment of PID (pelvic inflammatory disease) infections, uterosalfingeal washing is a very effective method. It is effective in PID infections which cannot be treated despite the use of high doses of antibiotics, and also in all three types of infections such as viral, bacterial, and fungal. The solution comprising ozonized oil or ozonide, which has no side effects but the healing, and provides infertility treatment, constitutes an effective washing solution even in a single use. It can be applied 2-3 times a week, or daily in case of heavy infections.

The Use of Solutions Comprising Ozonized Oil in Eve Diseases

The use of dilute solutions of ozonized oil or ozonides as eye drops in eye infections and allergic eye diseases both eliminates infection and provides anti-inflammatory and analgesic effects. It is used in the treatment of allergic eye diseases.

The solutions comprising nano or micron-sized ozonized oil of the invention can be used in the form of an eye drop or in various forms.

The Use of Solutions Comprising Ozonized Oil in Otorhinolaryngologic Diseases

The drops used in the external ear and middle ear infections and dermatitis cause the nesting of the fungal infection while eliminating the bacterial infection. If there is also dermatitis, a new preparation should be added to the treatment. The use of solutions of ozonized oil or ozonides as an ear drop allows the application of these multiple treatment methods at the same time. Thus, the treatment with a single product is provided instead of the one using several products. Dosages can be adjusted according to the intensity of infection. Use of solutions of ozonized oil or ozonides twice a day may be sufficient. Treatment of chronic dermatitis can take longer periods such as 1 month.

Sinusitis is a disease that is characterized by pain and throbbing in the acute or chronic phase, which is not possible to treat as it is known. Despite many treatments with antibiotics, it cannot be cured. Because it is not possible to reach the focus of the infection. This leads to unnecessary use of antibiotics. The studies carried on with the invention revealed that the regular use of ozonized oil or ozonide solution by reclining the head not only provides the removal of the infection but also activates the anti-inflammatory and analgesic effects by reaching into the sinus. Also, the modulation of the immune system solves issues relating to allergic rhinitis and permanently eliminates the sinusitis problem. The solutions of ozonized oil or ozonides at 40% concentration may be applied together with local anesthetics as it has a burning effect for the first 1-2 minutes. They can be combined with the decongestant or anti-inflammatory nasal drops. However, these are not generally needed. Even the initial application plays an effective role in the treatment. In these forms, preferably the form of saline water is used. However, it is important to use it regularly for 1 month, once a day and wait for the solution to enter the sinuses by keeping the head behind for 15 minutes following the application. The inside of the sinuses is completely cleaned due to daily application. Since it will be effective for allergic rhinitis, thus opening the nasal passages. Maintenance treatments include softening of the nasal mucosa, the use of nasal sprays comprising solutions of ozonized oil or ozonides which are prepared for anti-allergic and anti-inflammatory effects 2-3 times a day prevents the formation of infections by relieving the nasal mucosa. In these forms, preferably the form of saline water is used.

It also eliminates itchy infection picture in the nose caused by the flue. It reduces the crusts within the nose due to its oil content. There is no harm in long-term use.

The solutions comprising nano or micron-sized ozonized oils can be used in the form of a drop, spray or in various forms for the treatment of otorhinolaryngologic diseases. It can also be combined with the appropriate substances.

The Use of Solutions Comprising Ozonized Oil in Odontotherapy

In the case of oral infections such as stomatitis, gingivitis, etc., the application on gingiva or use as toothpaste is an effective treatment method.

The application of solutions comprising ozonized oils into dental cavities daily in doses such as 40% eliminates the infection by treating the cavity in a short period of time.

The injection of solutions comprising ozonized oil or ozonide in tooth extractions and dental treatments together with local anesthetics eliminates the infection and accelerates the healing process by increasing regional oxygenation. In the treatment of abscess, the injection of solutions comprising ozonized oil or ozonide into the abscess area prevents infection. In the post tooth extraction period and in the alveolitis treatment, local application of solutions comprising ozonized oil or ozonide into the tooth cavity prevents infection.

The solutions comprising nano or micron-sized ozonized oils can be used in oral care products in various forms such as gargle (mouthwash), toothpaste additive.

The Use of Solutions Comprising Ozonized Oil in Digestive System

In order to prevent superficial infection in throat infections, ozone products in the form of pastilles are used as mouth and throat antiseptics. The taste and effect of the pastille that contains ozonized oil or ozonide can be modified by adding antiseptics and sweeteners. Pastilles can be used three to five times a day. Since it does not contain antibiotics, it is a safe product that does not include toxic substances to be used in esophageal diseases. Pastilles comprising ozonized oil or ozonide doses of 5-10 (5000-10000 ppm) preferably made with ozonized oils comprising 15% ozone is suitable for the treatment.

Disposable packages with dilute solutions comprising ozonized oil or ozonides to be mixed in beverages can be prepared to be used by mixing into juices or other drinks. For this purpose, beverages comprising ozonized oil or ozonides can be prepared.

The use of solutions comprising stomach-soluble ozonized oils or ozonides 1-2 times a day for the treatment of gastric diseases, and particularly in the eradication of Helicobacter pylori is an effective method. In the eradication of Helicobacter pylori, at least 10 days of continuous use is required.

It is known that ozone is absorbed from the intestine and increases the oxygenation in the blood by passing systematically to the circulatory system. In particular, the capsules made with using injectable vegetable oils and dissolving in the intestine is used. Ozone is transported up to 160 mg with 1 gr oil. These doses of capsules with ozone of vegetable origin constitute a treatment method which is effective in the systemic circulation and non-tissue-damaging. Usually, ozonized oil or ozonide capsules comprising 50 (50000 ppm) mg ozone made with ozonized oils comprising 15% ozone are used. 1-2 capsules per day is appropriate.

The suppository comprising ozonized oil or ozonide prepared for use in the treatment of colon infections, colitis ulcerous, and Chron's disease, as well as autoimmune diseases, contains 320 mg ozone in 2 gr oil. The application of these suppositories 1-2 times a day reduces intestinal infections and increases intestinal oxygenation. It provides regulation of the immune system and heals autoimmune intestinal diseases. It also allows the passage of oxygen from the intestines to the bloodstream and creates systemic effects.

It provides alternative effects to major therapy. Also, the use of these suppositories is effective in the treatment of hemorrhoids, rectal fistulas, and fissures. The effectiveness of ozone oil made with Hypericum perforatum (St Johns-wort) is known in the treatment of hemorrhoids. The solutions comprising nano or micron-sized ozonized oil of Hypericum perforatum are used in the treatment of hemorrhoids in the form of suppositories. The solutions comprising nano or micron-sized ozonized oils obtained by ozonization of these oils in the form of suppositories are effective in the treatment of hemorrhoids, fistulas, fissures.

While the disposable solutions of ozonized oil or ozonide, which are formed in order to perform rectal enema application in sigmoid colon infections, ensure the disappearance of the infection, it also allows the passage of oxygen into the systemic circulation. Rectal irrigation with the disposable solutions of ozonized oil or ozonide is an effective method of treatment especially in patients with nutritional disorders and constipation issues in intensive care units.

The intraperitoneal injection of the solutions of ozonized oil or ozonide in peritonitis constitutes an effective method for the treatment of infection. The daily administration of solutions of 2% ozonized oil or ozonide of 20 ml (cc) by intraperitoneal injection is used to eradicate the infection and to create tissue regeneration.

The solutions comprising ozonized oil in nano or micron sizes of the invention can be produced in the form of capsules dissolving in different pH ranges, in suppository form or in various forms and in combinations with the appropriate substances.

The Use of Solutions Comprising Ozonized Oil in Vascular Diseases

It is known that streptokinase used for the opening of atheroma plaques in arteries also dissolves the thrombus plaque in the vessel in the acute phase. As a result of the arterial administration of solutions comprising ozonized oil formed as a result of ozonization of cholesterol, due to the adherence of ozone on the cholesterol to the atheroma plaque, the atheroma plaque is dissolved, and the vessel is opened. The administration of it with vitamin K or with other appropriate substances accelerates its effect.

In a preferred embodiment of the invention, it is possible to provide high ozonization of fats such as cholesterol, triglyceride, phospholipid, to be administrated intraarterially in combination with vitamin K in arterial vein occlusion, and to open the vessel with the antioxidant effect of ozonized oil products adhering to the vessel wall. The arterial injection of the solutions obtained by the ozonization of cholesterol adhere to the atheroma plaque and dissolves the atheroma plaque.

Ozone administration to the parenteral circulation is known to break down the surfactant in the alveoli. The use of particles of soybean oil in 100-150 micrometers in nutritional solutions ensures the intravenous use of oils. Similarly, as a result of the formation of the solutions of ozonized oil formed by ozonized soybean oil which is composed of ozonized oil or ozonide in small sizes up to 50 nanometers by ultrasonic digestion method, the slow release in parenteral solutions and the dissolution of ozone in the blood before it reaches the lung and is converted to free oxygen, a harmless agent is formed. For example, the administration of the solution of ozone oil comprising 100 mg ozone in 1 gram diluted preferably to 0.1-1% (100-1000 ppm) in 500 ml serum in 6 to 8 hours constitutes an effective parenteral solution. It can be effective in cardiac diseases as it increases cardiac health and coronary oxygenation.

The Use of Solutions Comprising Ozonized Oil in Skin Diseases

Ozone is widely used in skin diseases and cosmetics as ozone oil and ozonized water. Ozone oils in various doses obtained by the ozonization of oils depending on the ozonization process are still used. Since the solutions comprising ozonized oil of the present invention contain ozonized oil in the range of 1 nanometer to 400 microns relative to other dense ozonized oils, preparations that are much faster to absorb from the skin are formed. Besides, it can be used together with vitamins A, D, E, K as particles in nanoscale or micron sizes and accordingly increase their absorption from the skin. Due to nano or micron-sized particles, it facilitates the absorption of many substances from the skin.

Solutions comprising ozonized oil in nano and micron sizes can be used for dermatitis, acne, allergic dermatitis, anti-aging and wrinkle prevention.

The subcutaneous administration of the injectable agents created as a result of the ozonization of soybean oils and alpha lipoic acid used for mesotherapy is a novel method of the invention. The lipolytic properties of ozone are known. Administration of ozone gas for mesotherapy is a fat-burning method. Alpha lipoic acid is also used for the same purpose. The efficiency of mesotherapy is increasing with the injection of nano or micron-sized particles formed by the ozonization of alpha lipoic acid under the skin. It can also be combined with other mesotherapy agents such as phosphatidylcholine. As a result of lipolysis, the fat transferred to the liver is ozonized and the effect of fat on the liver is reduced. Can be applied 1 to 2 times per week.

In hair mesotherapy, the subcutaneous application of the solutions of ozonized oil or ozonide increases regional oxygenation and increases the nourishment of hair follicles. It can be combined with other hair mesotherapy agents such as vitamin E and minoxidil. Can be applied 1 to 2 times per week. In the studies conducted within the scope of the invention, the solutions of the present invention used for hair treatments in combination with fat-soluble vitamins, water-soluble vitamins, saw palmetto, ginseng combined with fat-soluble forms of vegetable extracts, have been found to enhance the efficacy of the samples.

The use of solutions comprising nano or micron-sized ozonized oils in facial mesotherapy increases collagen synthesis by increasing oxygenation under the skin. It increases the regeneration ability of the tissue and reduces the formation of wrinkles. In the studies conducted within the scope of the invention, the solutions of the present invention used for skin treatments in combination with fat-soluble vitamins, water-soluble vitamins, hyaluronic acid, ginseng combined with fat-soluble forms of vegetable extracts, have been found to enhance the efficacy of the samples.

The solutions comprising nano or micron-sized ozonized silicone oil and other solutions comprising ozonized oil are used for facial mesotherapy. Due to the invention, the formation of spots is reduced with the removal of the toxic substances accumulated in subcutaneous tissues from the environment by the oxidation of free radicals. Ozone gas injection into subcutaneous tissues is known to reduce blotches. The injection of solutions comprising ozonized oils into subcutaneous tissues 1-2 times a week constitutes an effective method of treatment. The solutions comprising nano or micron-sized ozonized oils can be combined with other injectable products such as Vitamin E, Vitamin A, vitamin C, hyaluronic acid. The use of it combined with Vitamin K, with Vitamin C, and with hyaluronic acid constitutes an effective method in the treatment of purple eyes.

The use of solutions comprising ozonized oils in the content of the shampoos makes the skin healthier and prevents hair loss.

The Use of Solutions Comprising Ozonized Oil in Cancer Treatment

It is known that the oxygen demand and the peripheral vascularity of cancer cells increases. It is also known that solutions of ozonized oil and ozonide contain high oxygen. Accordingly, it is possible to produce a specific chemotherapeutic treatment due to the fact that Nano or micron-sized ozone oils can easily reach the cancerous cells following the administration of solutions comprising ozonized oils to cancerous tissues together with chemotherapeutic drugs. Regional administration limits the effects of systemic chemotherapy. In the case of intra-cavity tumors such as bladder tumors, intestinal tumors, peritoneal tumors, the solutions comprising ozonized oils can be injected into the cavity together with the chemotherapeutic agent.

The administration of the solutions of ozonized oil to the cancerous tissue and its periphery locally at high concentrations and high doses in cancer treatment makes cancer tissue hypoxic. The use of the poppy oil injection through the hepatic artery in liver tumors constitutes effective treatment. Poppy oil is ozonized in nano or micron sizes, given in the same way, so that, its entry into the cancer cell is facilitated and its effect can be increased.

In addition to cancer prevention and chemotherapy, regular injections of the solutions comprising ozonized oil in nano or micron sizes create the antioxidant effect. In the treatment of cancer, regional injection of the solutions comprising ozonized oil may put the cancerous tissue in anoxia at high doses.

The Use of Solutions Comprising Ozonized Oil in Veterinary Medicine

The solutions comprising ozonized oil in nano or micron sizes can be used for the purposes of protecting animal health, for the eradication of bacteria, viruses, and fungi, as well as for increasing their healthy development and reproductive capabilities.

In order to reduce the cause of death by infection of the animals in the developmental process, the intramuscular injection of solutions comprising ozonized oil once a week is an effective method in preventing animal diseases. It reduces costs by reducing the use of antibiotics.

The mixing of it to the drinking waters of livestock animals prevents the formation of bacteria and parasites in the intestine as well as enhances the health of the animals.

The solutions comprising nano or micron-sized ozonized oils can be used both inside the fistula and in the form of a regional injection for treating mastitis in veterinary medicine.

The solutions comprising nano or micron-sized ozonized oils can be applied into the vagina in vaginal infections and applied to the oral areas in oral and nasal infections as effective treatment methods in veterinary medicine.

The solutions comprising nano or micron-sized ozonized oils can be applied to the cavity and periphery of the abscess for the abscess treatment in veterinary medicine.

The solutions comprising nano or micron-sized ozonized oils can be applied as intra-articular injections in the treatment of joint diseases and as regional injections in treating regional pain in veterinary medicine, just like humans.

The Use of Solutions Comprising Ozonized Oil in Food Industry

In the current technique, paraffin and similar oils are squeezed onto the fruit and vegetables to make them look brighter. Such processes, especially in the digestive system, have many harms in terms of human health. The use of solutions comprising nano or micron-sized ozonized oils obtained with the ozonization of vegetable oils in diluted doses for the same purpose prolongs the shelf life of fruits and vegetables. Due to the oil content, these solutions make them look bright and fresh. Due to water content, drying of them is prevented. In addition, it has said positive effects on human health. In the studies carried out with the solutions of the invention, the solutions comprising ozonized oil in nano or micron size forms a thin layer on the sprayed surfaces of fruit and vegetables. This layer prevents the growth of bacteria and fungi on fruit and vegetables and delays the process of decay.

As known, extending the shelf life of fruits and vegetables is an important issue in exports. In the process of exporting the tomato to another country by land transports, various reasons such as delays in customs, deterioration in cold air systems sometimes cause the goods to become defective and not accepted when they reach their destination. When the solutions comprising ozonized oil in nano or micron sizes are sprayed on the fruits and vegetables and they are placed in crates, due to the thin layer of ozonized oil or ozonide formed on fruits and vegetables, they are not affected by the damage in the place of contact, and since the formation of bacteria and fungi is prevented, the shelf life is much longer. In one embodiment of the invention, 1 liter of ozonized oil or ozonide solution preferably made with ozonized oil comprising 10% ozone comprising 40 g ozone is mixed into 100 liters of water. The mixing process conducted with a mixer ensures better dispersal of the solution.

During the packaging, spraying can be applied also on the line. In a study conducted with the solutions of the invention, a group of fruits and vegetables were sprayed with a solution comprising preferably 1% ozonized oil and another group of fruits and vegetables were not sprayed with a solution comprising ozonized oil. These fruits and vegetables were kept at room temperature. 10 days later, it is observed that while the wrinkling and decay in the fruits and vegetables that are not sprayed is unacceptable, the fruit and vegetables sprayed with the solution comprising ozonized oil did not lose their healthy images. The percentage of the solution comprising ozonized oil used in the study as %1 is not a limiting value. This ratio can be increased. If this ratio is increased, the durability of fruits and vegetables also increases in direct proportion. There is no harm in the consumption of these vegetables and fruits due to the use of the solutions comprising ozonized oil of vegetable origin. On the contrary, by consuming this way, the beneficial effects of ozonized oils on the digestive system are utilized. To eliminate the ozone gas, it is enough to heat the foods.

Similarly, the packaging of meat by passing through the ozone gas is known to increase shelf life by two times. Spraying of the solutions comprising ozonized oil in nano or micron sizes formed by ozonization of vegetable oils in a thin layer in dilute doses during the packaging of white meat and red meat ensures that the meats seem fresh and alive and prevents them from drying due to its water content. It also prevents bacterial, viral and fungal contamination. And thus, prolongs the shelf life. There is no harm in the consumption of these foods. As is known, ozonized oils or ozonides have healing effects on the digestive system when swallowed. The use of solutions comprising ozonized oil in nano or micron sizes as stabilizers in order to prevent decay of the packaged foods and beverages makes it both harmless and a useful additive for the digestive system and general body health.

The Use of Solutions Comprising Ozonized Oil in Chicken Breeding

The benefits of the use of ozone pills to human health are known and they have been used for many years. The effects are profound, especially in the treatment of stomach and intestinal infections. It is also effective in parasitic and fungal infections. It also improves health and provides oxygen to the body. The same applies to animal health. In particular, it is very common for chickens living in poultry farms with dense populations in congestion to infect each other and cause great outbreaks. This leads to mass deaths. Mixing the solutions comprising ozonized oil in nano or micron sizes to the drinking water of chicken prevents the infections as well as enhances the health of the chicken. The calculation of solutions comprising ozonized oil in nano or micron sizes that are added to the drinking water of chickens can be made according to the daily human dose/kilos.

At the same time, ozone released into the environment helps eliminate the bad odors in the chicken farm and reduces the number of bacteria spread in the air. For example, there is 40 mg ozone in the ozone capsules prepared for humans. Assuming that the chickens are 2 kg on average, mixing the 1 mg dose into drinking water is sufficient. In a preferred embodiment of the invention, the solution comprising ozonized oil comprising 40 g of ozone in 1 liter is 40000 mg, the solution comprising ozonized oil comprising 1 liter of ozone per day can be added to the amount of daily water needed by 40000 chickens. The mixing process conducted with a mixer ensures better dispersal of the solution.

The use of solutions comprising ozonized oil in nano or micron sizes in chicken breeding allows chickens to produce healthier eggs and protects them from infectious diseases. In addition, it reduces the use of antibiotics is in chicken breeding.

The Use of Solutions Comprising Ozonized Oil in Plant Spraying

Because of the dilution of the solutions comprising ozonized oil and the prolonged usage life of it due to its oil content, it becomes an effective method for the spraying of plants. The thin layer on vegetables and fruits formed by the spraying with ozonide solution remains for 3 weeks and nourishes the fruit peel by preventing the formation of rot from the invisible fine holes created by pests on vegetables and fruits. Considering that the duration of stay on the plant according to weather conditions is 2-3 weeks, the application of solutions comprising ozonized oil with 3 weeks intervals during the development period of vegetables and fruits can make important contributions to plant care.

Because it is not harmful to health like pesticides and it is beneficial if it is swallowed, it does not cause any harm both during spraying and in case of ingestion. Inhalation during spraying may cause harm and burn in the eyes. Therefore, a mask should be used during spraying. However, there is no harm in swallowing some. In a preferred embodiment of the invention, it is sufficient to add and spray the ozone loaded solution comprising 1 liter of ozonized oil to 100 liters of water, mixed with the help of the mixer in a ratio of 1000-5000 ppm (mg/L) in doses determined by the studies depending on the plant. Solutions comprising ozonized oil in nano or micron sizes are suitable for use in organic farming.

Plant spraying is costly and has a negative effect on human health due to the spread of chemical wastes to nature. The application made with solutions comprising ozonized oil is completely natural, it does not harm the plant and prevents harmful bacteria and fungi from forming cavities.

The Use of Solutions Comprising Ozonized Oil in Conservation of Ornamental Plants

Because of the density of the paraffin as oil being applied to protect the leaves of ornamental plants and make them look brighter and while creating a bright picture on the leaf, it prevents the leaf from getting air. Prolonged use of it causes fungus formation on the leaf.

The use of solutions comprising diluted ozonized oil as a spray increases the lifetime of the plant due to its low oil ratio and its oxygen secretion ability, and with its small amount of oil content, it makes the leaf look brighter by creating a thin layer on it. In a preferred application of the invention, spraying at intervals of 10 days is sufficient for the plant to appear bright. For this, the solutions of 1% (1000 ppm) are sufficient. Solutions comprising ozonized oil in nano or micron sizes prevent fungus and bacteria formation on the plant.

In order to protect ornamental plants and improve the health of the leaf, the application of ozone-comprising oil solutions to the leaves and the trunk/body provides an enhancing effect on plant health.

The Use of Solutions Comprising Ozonized Oil in Chemical Industry

It is known that ozone, which is a powerful disinfectant, is used for water disinfection, in disinfection of storages and hygienic areas.

Solutions comprising ozonized oil in nano or micron sizes in combination with dishwasher detergents and cleaning agents increase the disinfectant effect of detergents and prevent the formation of contact dermatitis by reducing the toxic effects of the cleaning agents on the skin.

In a preferred application of the invention, mixing solutions comprising 3-5% ozonized oil in a variety of doses of 1000-5000 ppm (mg/L) during the preparation of detergents increases the disinfectant effect of the detergent and provides therapeutic effects against the adverse effects of detergents on the skin. These ratios can also be changed according to the desired duration of efficacy.

Solutions comprising ozonized oil in nano or micron sizes can be added to cleaning materials and used in various forms and for various purposes.

The Use of Solutions Comprising Ozonized Oil in Hygienic Wipes

Disposable wipes used for hygiene can be divided into groups such as baby wet wipes, daily wet wipes, intimate cleansing wipes, or wipes for wound care, makeup removal, face and skin care, or wipes created for the elimination of warts. The use of ozone as a disinfectant in the solutions of the invention, its tissue healing properties and its ability to keep the tissue moist via the oils provides the multi-purpose use of the solutions comprising ozonized oil in nano or micron sizes. The solutions of the invention have no side effects compared to other products comprising toxic substances. On the contrary, it has tissue healing properties. Solutions comprising ozonized oil in nano or micron sizes can be used in disposable wipes alone, preferably at a rate of %1-5 (1000-5000 ppm).

Solutions comprising ozonized oil in nano or micron sizes prevent rash formation on babies due to its oil content. Therefore, there is no need to use other creams. It can be used with other antibacterial agents.

For the purpose of genital cleansing, solutions comprising ozonized oil in nano or micron size at the high rates such as %10-20 (10000-20000 ppm) are used to prevent the formation of fungi and viruses. The use of it before or after sexual intercourse prevent contaminations by contact. It also improves the healing of the tissue. It is also suitable to use intra-vaginal solutions in combination with solutions comprising ozonized oil in nano or micron sizes.

In accordance with the descriptions above, the invention relates to the use of solutions of ozonated oil comprising nano or micron sizes comprising 10-100 000 mg/L (ppm) ozone gas as a disinfectant, preservative or food supplement in the food industry; as a disinfectant, repellent, feed-fattening contribution in the livestock industry; in pharmaceutical compositions for treatment of diseases in the health industry, especially in dermatology, otorhinolaryngology, eye diseases, oral-dental diseases, digestive system, urology, gynecology, orthopedics, circulatory system diseases, ozone therapy and mesotherapy; in the production of personal care products used in the cosmetics industry, especially in the skin, mouth, hair care; in the production of detergents and disinfectant products in the chemical industry and as an additive for the petroleum products; in textile industry as bleach, disinfectant or anti-bacterial textile chemicals; as a preservative and repellent in end products in agricultural industry, as an insecticide, pesticide, fungicide, virucide, and bacteria killer in cultivated products, as a plant and soil disinfectant, and as a fertilizer in agriculture or soilless agriculture.

To perfect the invention according to the uses above, the following products which are obtained from solutions comprising nano or micron-sized ozonized oil comprising 10-100 000 mg/L (ppm) ozone gas are within the scope of the invention:
A nasal solution comprising 10 to 5000 mg/L of ozone gas, preferably with ozonized oil comprising 5% ozone gas by weight, and preferably with physiological saline solution, having a greater oil density, to provide lubrication of the nasal mucosa. Other forms of water can also be used in this solution. Here, “ozonized oil comprising 5% ozone gas” expression means that every 100 grams of ozonized oil used in the preparation of the solution comprise 5 grams of ozone gas. The term “solution comprising 10 to 5000 mg/L of ozone gas” means that each 1-liter solution can contain ozone gases from 10 to 5000 mg.
A nasal solution comprising 500 to 20000 mg/L of ozone gas, preferably with ozonized oil comprising 15% ozone gas by weight, and preferably with physiological saline solution, having a lower oil density, to be used in the treatment of sinusitis. Other forms of water can also be used in this solution.
An injectable solution with ozonized oil comprising preferably 10% ozone gas by weigh and ozone gas from 10 to 10000 mg/L, preferably distilled water, provided in a ready-made glass injector, silicone lid, glass vial, silicone cap, ampoule, ozone-resistant serum bottles, and plastic bags due to the ozone interaction for subcutaneous, intraarticular, intramuscular, intra-peritoneal mesotherapy applications to ensure the standardization of these therapies.
A hair solution of 1000 to 10000 mg/liter of ozone gas, comprising ozonized oil comprising preferably 15% ozone gas together with vitamins, minerals, and active ingredients, having the more ozone density by weight since oil density is not desired to be high, in order to improve hair and grow new hair.
A bee solution of 1000 to 50000 mg/liter of ozone gas, obtained by ozonized oil comprising 10% ozone gas by weight combined with thymol or plant extracts and sugared water, which eliminates the possibility of oil intensity damaging bees in bee treatments.
A solution for fruit and vegetables obtained by ozonized oil comprising 5% ozone gas by weight, comprising 100 to 5000 mg/liter ozone gas, or comprising 100 to 10000 mg/liter ozone gas prepared from ozonated oil comprising 15% ozone gas to provide a drier image in order to increase the shelf life of dried fruit and vegetables, to keep them storable for a longer time with its oil content and to make them appear bright,
An eye drop comprising a solution comprising ozone gas of 10-5000 mg/liter, obtained by ozonized oil comprising 10% ozone gas by weight to be used for eye health.
An eardrop comprising a solution comprising ozone gas of 1000-10000 mg/liter, prepared from ozonized oil comprising 5% ozone gas by weight to be used in the treatment of intensive fungal infections in ear health.
A dermatological solution comprising ozone gas of 100-30000 mg/liter, obtained by ozonized oil comprising 10% ozone gas by weight to be used in the treatment of dermatitis in allergic diseases.
A serum solution comprising ozone gas of 10-4000 mg/liter, obtained by ozonized oil comprising 10% ozone gas by weight presented in silicone bags or glass bottles to be used in parenteral injections.
An injectable solution with ozonized oil comprising preferably 10% ozone gas by weight in combination with fat-soluble vitamins, hyaluronic acid, and active ingredients and ozone gas from 10 to 5000 mg/L, provided in the form of an ampoule, vial and glass syringe for subcutaneous, intraarticular, intramuscular, intra-peritoneal mesotherapy applications.
A dermatological solution presented in the form of a spray, comprising ozone gas of 2000-70000 mg/L, prepared from ozonized oil comprising 15% ozone gas by weight to be used in the treatment of wounds.
A dermatological solution presented in the form of a spray, comprising ozone gas of 2000-70000 mg/L, prepared from ozonized oil comprising 15% ozone gas by weight in combination with disinfectants comprising iodine, betadine, silver ions to be used in the treatment of wounds.
A dermatological solution presented in the form of a spray, comprising ozone gas of 100-70000 mg/L, obtained by ozonized oil comprising 15% ozone gas by weight to be used in skin rejuvenation.
A dermatological solution presented in the form of a spray, comprising ozone gas of 100-10000 mg/L, prepared from ozonized oil comprising 15% ozone gas by weight in combination with skin rejuvenation and stain-free products to be used in skin rejuvenation and stain treatments.
A urological solution to be presented in the form of a one-use lubricant in silicone or an injector for an antiseptic probe, comprising ozone gas of 100-2000 mg/L, prepared from ozonized oil with high oil rate comprising 2-5% ozone gas by weight to be used in urology.
A urological solution to be presented in the form of injection for an antiseptic bladder, comprising ozone gas of 100-5000 mg/L, prepared from ozonized oil comprising 15% ozone gas by weight to be used in urology.
A vaginal solution to be presented in the form of disposable packages, bags, or syringe, comprising ozone gas of 100-10000 mg/L, obtained by ozonized oil comprising 15% ozone gas by weight to be used in vaginal antisepsis.
An injectable solution to be presented in the form of injection for use in infertility tubs, comprising ozone gas of 100-5000 mg/L, prepared from ozonized oil comprising 10% ozone gas by weight.
An injectable solution to be presented in the form of antiseptic ampoules, injections and vial forms, comprising ozone gas of 100-10000 mg/L, prepared from ozonized oil comprising 10% ozone gas by weight to be used in-cavity and in canal treatment in dental health.
Stomach-soluble, small intestine-soluble, and lower intestine-soluble capsules or gels, including the solution comprising ozone gas of 50000-70000 mg/L, prepared from ozonized oil comprising 15% ozone gas by weight to be used in digestive system health.
A single use rectal solution comprising ozone gas of 100-10000 mg/L, prepared from ozonized oil comprising 10% ozone gas by weight to be used in the treatment of lower intestine diseases.
Suppositories solidified with solid paraffin or witepsol with solutions comprising ozone gas of 10000-70000 mg/L, prepared from ozonized oil comprising 15% ozone gas by weight to be used in the treatment of lower intestine diseases.
Toothpaste including the solutions comprising ozone gas of 500-2000 mg/L, prepared from ozonized oil comprising 10% ozone gas.
A gargle (mouthwash) in single use forms or in bottles, comprising solutions comprising ozone gas of 100-5000 mg/L and preferably sweetener, prepared from ozonized oil comprising 10% ozone gas.
Pastilles comprising solutions comprising ozone gas of 5000-7000 mg/L and preferably sweetener, prepared from ozonized oil comprising 15% ozone gas.
A condensed solution to be used as diluted, comprising ozone gas of 100-70000 mg/L, prepared from ozonized oil comprising 10% ozone gas by weight to be used as a feed additive in animal health.
An injectable solution presented in the form of injectors, vials, and ampoules to be applied by intramuscular, intraarticular, intravenous, subcutaneous, intravaginal, intraperitoneal injections, comprising ozone gas of 100-10000 mg/L, prepared from ozonized oil comprising 10% ozone gas by weight to be used for the treatment of animal health.
A single use rectal solution comprising ozone gas of 100-5000 mg/L, prepared from ozonized oil comprising 15% ozone gas by weight to be used to protect animal health.
Suppositories solidified with solid paraffin or Witepsol with solutions comprising ozone gas of 1000-70000 mg/L, prepared from ozonized oil comprising 15% ozone gas by weight to be used to protect animal health.
A condensed solution presented in the form of a spray to be diluted, comprising ozone gas of 1000-70000 mg/L, prepared from ozonized oil comprising 10% ozone gas by weight to be used as a repellant, insecticide, and disinfectant in poultry farms, barns and environments like stockyards.
A solution presented in the form of a spray or a solution, comprising ozone gas of 1000-70000 mg/L, prepared from ozonized oil comprising 10% ozone gas to be used for wound and skin care, disinfection, hygiene purposes to protect animal health.
A solution presented in the form of a spray or a solution, comprising ozone gas of 1000-10000 mg/L, prepared from ozonized oil comprising 10% ozone gas to be used for wound and skin care, disinfection, hygiene purposes in human healthcare.
A solution comprising ozone gas of 100-30000 mg/L, preferably prepared from ozonized oil comprising 10% ozone gas to be used in the food industry for the protection of meat, fruit, and vegetables, for the elimination of pesticide and for antibacterial effects by immersion or spraying.
A solution comprising ozone gas of 100-5000 mg/L, preferably prepared from ozonized oil comprising 10% ozone gas to be used in the food industry to prevent deterioration of dried foods, pulses and to provide repellant effects by immersion or spraying.
A solution comprising ozone gas of 10-2000 mg/L, preferably prepared from ozonized oil comprising 5% ozone gas to be used as a disposable beverage or as in a combination with other drinks and sweeteners.
Disposable wipes or wet wipes comprising the solutions of ozone gas by 500-100000 mg/L, preferably prepared from ozonized oil comprising 10-15% ozone gas.
Room, surface, or device cleaning agents or detergents comprising solutions of ozone gas by 1000-100000 mg/L, preferably prepared from ozonized oil comprising 10-15% ozone gas.
A solution for plant spraying presented in a condensed form to be diluted or as a spray, comprising ozone gas of 100-20000 mg/L, prepared from ozonized oil comprising 10% ozone gas by weight to be used to provide anti-bacterial, anti-fungal, the anti-parasitic and repellant effects in plants.
A fertilizer solution comprising ozone gas of 100-10000 mg/L, prepared from ozonized oil comprising 10% ozone gas to be used in soilless agriculture.
A seed solution comprising ozone gas of 100-20000 mg/L, prepared from ozonized oil comprising 10% ozone gas to be used to improve seeds, to provide the anti-bacterial effects in seed cultivation, and to remove soil pathogens for germination purposes in agriculture.
A solution for plants presented in the form of a spray, comprising ozone gas of 100-5000 mg/L, prepared from ozonized oil comprising 5% ozone gas to be used to provide both brightness in ornamental plants and increase plant health.
A fuel additive solution comprising ozone gas of 100-70000 mg/L, prepared from ozonized oil comprising 15% ozone gas to be used to improve vehicle performance when mixed in petroleum products.

1. Solutions comprising ozonized oil in nano or micron sizes, and comprising oil, water, emulsifier and 10-100 000 mg/L ozone gas, wherein the solution comprises 1-70% of oil by weight, 0.001-10% of ozone gas by weight, 10-97.9% of water by weight, and 1-50% of at least one emulsifier by weight, and
comprising ozonized oil with particle size of 1 nanometer-400 micron.

2. (canceled)

3. The solutions according to claim 1, wherein said oil comprises an individual or combinations selected from the group comprising of soybean oil, St. John's Wort oil, sesame oil, palm oil, poppy oil, soy lecithin, cholesterol, b-sterol, triglyceride, olive oil, fish oil, sunflower oil, castor oil, saffron oil (safflower), coconut oil, triglyceride derivatives, tributyrin, tricaproin, tricaprylin and paraffin (mineral oil), ethyl oleate, methyl oleate.

4. (canceled)

5. The solutions according to claim 1, wherein said water is distilled water, saline water, sugared water, mineral water, deionized water, demineralized water, spring water, saline solution or physiological saline solution.

6. The solutions according to claim 1, wherein said emulsifier comprises an individual or combinations selected from the group comprising of lecithin formed from egg yolk, glycerin, lysophospholipid, polyethylene glycol (PEG), phosphatidylethanolamine (PEG-PE), pluronic, Polysorbate 20, Polysorbate 80, or an emulsifier suitable pharmacologically or chemically.

7. (canceled)

8. The solutions according to claim 1, further comprising water and/or oil-soluble vitamins, minerals, hyaluronic acid, plant extracts or active ingredients, essential oils or thymol.

9. A method to produce the solutions comprising ozonized oil in nano or micron sizes according to claim 1, characterized by comprising the process steps of:
A. Ozonization of selected oils until they absorb the specified amount of ozone gas, wherein comprising weighing the oil before ozonization by precision scale, then weighing again after ozonization, thus obtaining amount of ozone absorbed in the oil in milligrams;
B. Mixing of water with at least one emulsifier, wherein the temperature of the mixture is 10-15° C. above the melting temperature of the oil depending on the type of oil used;
C. Addition of ozonized oils to the mixture comprising water and at least one emulsifier and blending of them in the mixer with the cutting blades, wherein the temperature of the mixture comprising water, at least one emulsifier, and the ozonized oil is 10-15° C. above the melting temperature of the oil depending on the type of oil used;
D. Spray cooling of the solution.

10. (canceled)

11. (canceled)

12. (canceled)

13. (canceled)

14. (canceled)

15. Use of the solutions comprising ozonized oil in nano or micron sizes according to claim 1:
as a disinfectant, preservative or food supplement in the food industry;
as a disinfectant, repellent, feed-fattening contribution in the livestock industry;
in pharmaceutical compositions for treatment of diseases in the health industry, especially in dermatology, otorhinolaryngology, eye diseases, oral-dental diseases, digestive system, urology, gynecology, orthopedics, circulatory system diseases, ozone therapy and mesotherapy;
in the production of personal care products especially used for skin, mouth, hair care in the cosmetics industry;
in the production of detergents and disinfectant products or as an additive for the petroleum products in the chemical industry;
as bleach, disinfectant or anti-bacterial textile chemicals in the textile industry;
as a preservative and repellent in end products in the agricultural industry, as an insecticide, pesticide, fungicide, virucide, and bacteria killer in cultivated products, as a plant and soil disinfectant, and as a fertilizer in agriculture or soilless agriculture.

16. A nasal solution according to claim 1, comprising 10-5000 mg/L of ozone gas; obtained by ozonized oil comprising 5% ozone gas by weight and physiological saline solution preferably; having a greater oil density to provide lubrication of the nasal mucosa.

17. (canceled)

18. An injectable solution according to claim 1, ensuring the standardization of the therapies for subcutaneous, intraarticular, intramuscular, intra-peritoneal mesotherapy applications; comprising 10-10000 mg/L of ozone gas; obtained by ozonized oil comprising 10% ozone gas by weight and distilled water preferably; provided in a ready-made glass injector, silicone lid, glass vial, silicone cap, ampoule, ozone-resistant serum bottles, and plastic bags due to ozone interaction.

19. A hair solution according to claim 1, to improve new hair growth comprising 1000-10000 mg/liter of ozone gas; obtained by ozonized oil comprising 15% ozone gas by weight together with vitamins, minerals, and active ingredients preferably; having more ozone density by weight since oil density is not desired to be high.

20. A bee solution according to claim 1, which eliminates the possibility of oil intensity damaging bees in bee treatments, comprising 1000-50000 mg/liter of ozone gas; obtained by ozonized oil comprising 10% ozone gas by weight combined with thymol or plant extracts and sugared water preferably.

21. (canceled)

22. An eye drop solution according to claim 1, for eye health comprising 10-5000 mg/liter ozone gas, obtained by ozonized oil comprising 10% ozone gas by weight preferably.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A single use rectal solution according to claim 1, for use in treatment of lower intestine diseases, comprising 100-10000 mg/L of ozone gas; obtained by ozonized oil comprising 10% ozone gas by weight preferably.

38. (canceled)

39. (canceled)

40. (canceled)

41. (canceled)

42. (canceled)

43. (canceled)

44. (canceled)

45. (canceled)

46. (canceled)

47. (canceled)

48. A solution according to claim 1, used for wound and skin care, disinfection, hygiene purposes in human healthcare, presented in the form of a spray or a solution, comprising 1000-10000 mg/L of ozone gas, obtained by ozonized oil comprising 10% ozone gas by weight preferably.

49. (canceled)

50. A solution according to claim 1, used in the food industry to prevent deterioration of dried foods, pulses and to provide repellant and insecticide effects by immersion or spraying; comprising 100-5000 mg/L of ozone gas, preferably obtained by ozonized oil comprising 10% ozone gas by weight.

51. (canceled)

52. (canceled)

53. (canceled)

54. A plant solution according to claim 1, used to provide antibacterial, antifungal, the antiparasitic and repellant effects in plants, presented in a condensed form proper to use for spraying by diluting with water, comprising 100-20000 mg/L of ozone gas, obtained by ozonized oil comprising 10% ozone gas by weight preferably.

55. (canceled)

56. (canceled)

57. (canceled)

58. (canceled)
